(19) United States
(12) Patent Application Publication
(10) Pub. No.: US 2010/0040584 A1
(43) Pub. Date: Feb. 18, 2010

(54) METHODS FOR PROMOTING NEOVASCULARIZATION

(75) Inventors: Juan M. Melero-Martin, Newton Highlands, MA (US); Joyce E. Bischoff, Weston, MA (US)

Correspondence Address: DAVID S. RESNICK, NIXON PEABODY LLP, 100 SUMMER STREET, BOSTON, MA, 02110-2131, US

(73) Assignee: CHILDREN'S MEDICAL CENTER CORPORATION, Boston, MA (US)

(21) Appl. No.: 12/520,185

(22) PCT Filed: Dec. 19, 2007
(86) PCT No.: PCT/US07/88110

§ 371 Date: Jun. 19, 2009

Related U.S. Application Data
(60) Provisional application No. 60/875,737, filed on Dec. 19, 2006.

(51) Int. Cl. A61K 35/12; A61P 9/00

(52) U.S. Cl. 424/93.7

(57) ABSTRACT

The success of tissue engineering and therapeutic neovascularization depends on the development of a microvascular network. The present invention provides methods for promoting neovascularization in tissue engineering constructs, tissue repair, and wound healing comprising endothelial and mesenchymal progenitor cells.

### CROSS REFERENCE TO RELATED APPLICATIONS

This application claims benefit of U.S. Provisional Patent application No. 60/875,737 filed Dec. 19, 2006, the contents of which are incorporated herein by reference in its entirety.

### GOVERNMENT SUPPORT

This invention was made with Government support under Grant No.: W81XWH-05-1-0115 awarded by the Department of Defense. The Government has certain rights in the invention.

### BACKGROUND OF THE INVENTION

Tissue engineering (TE) holds a great promise as a new approach for creating replacement tissue to repair congenital defects or diseased tissue. One strategy is to seed the appropriate cells on a biodegradable scaffold engineered with the desired mechanical properties, followed by stimulation of cell growth and differentiation in vitro, such that, on implantation in vivo, the engineered construct undergoes remodeling and maturation into functional tissue. Examples of this approach include blood vessels, cardiovascular substitutes, bladder, skin, and cartilage where autologous vascular cells have been used for this purpose without immune rejection.

Despite advances in this field, TE still faces major constraints. Most tissue in the human body require a functional microvascular network for the efficient delivery of oxygen and nutrients and removal of waste materials. One big barrier in organ and tissue engineering is neovascularization of the engineered tissue. Currently, there are no TE constructs presently available that have an inherent microvascular bed ready to be connected to the host vascular system. Consequently, tissues implanted with a volume greater than 2 to 3 mm cannot obtain appropriate provision of nutrients, gas exchange, and elimination of waste products since all these mechanisms are limited by the diffusion distance. Without the development of the microvascular network, the engineered tissue is not sustainable and dies with time. Therefore, neovascularization of engineered tissues and organs is a major challenge of TE.

### SUMMARY OF THE INVENTION

Embodiments of the present invention provides a method of promoting neovascularization in a tissue in need thereof comprising contacting the tissue with a composition comprising an enriched population of isolated endothelial progenitor cells and an enriched population of isolated mesenchymal progenitor cells, wherein the endothelial progenitor cells and mesenchymal progenitor cells induce the formation of new blood vessels with functional connections to the host vasculature. The progenitor cells that are in contact with the tissue can be from the same composition or separate composition.

In one embodiment, the method of promoting neovascularization occurs in tissue engineering constructs. Tissues need neovascularization to receive oxygen and nutrients for growth and maintenance. Neovascularization is also needed for the removal of metabolic waste that can be toxic if left to accumulate in the tissue. Any tissue engineered construct greater than 2 mm thick requires neovascularization for viability and maintenance after implantation in the host.

In one embodiment, in order to promote neovascularization in TE constructs, endothelial progenitor cells and mesenchymal progenitor cells are used to seed a TE scaffold. For example, these progenitor cells can be seeded along with other cell types that are normally used for making the tissue engineered construct. For example, embryonic stem cells, and tissue-derived cells such as keratinocytes, cardiac progenitors, and hepatocytes.

In one embodiment, the method of promoting neovascularization occurs in a tissue that is ischemic. Such neovascularization occurs in therapeutic vasculogenesis. Therapeutic vasculogenesis is useful for promoting tissue repair and wound healing. Promoting neovascularization at the site of injury or damage can help speed the repair. Ischemic tissues and organs having reduced blood flow can also benefit from therapeutic vasculogenesis using the invention. In one embodiment, the ischemic tissue includes, for example, the heart, skin, adipose tissue, muscle, brain, bone, liver, lungs, intestines, legs, limbs and kidneys. The composition containing the progenitor cells is contacted by direct injection to the ischemic tissue or to healthy tissue adjacent to the ischemic tissue. The composition containing endothelial progenitor cells can be delivered alone or mixed with mesenchymal progenitor cells prior to delivery. Alternately, the composition containing mesenchymal progenitor cells can be delivered alone or mixed with endothelial progenitor cells prior to delivery.

Promoting neovascularization can also stimulate wound healing. In certain instances, wound healing is impaired due to a variety of medical conditions such as congestive heart failure, poor circulation, obesity, lymphatic obstructions and diabetes. For example, pressure ulcers, leg ulcers, abrasions, lacerations, incisions, donor sites and second degree burns on infected wounds, surgical incisions and traumatic wounds can all benefit from neovascularization. The composition comprising an enriched population of isolated endothelial progenitor cells and an enriched populations of isolated mesenchymal progenitor cells are delivered directly by injection to the tissue needing repair, to the wound, and/or to the healthy tissue adjacent to the wound. In another embodiment, the composition is delivered on a wound dressing material which is then placed on the wound. As noted above, in an alternate embodiment, the enriched populations of isolated endothelial progenitor cells and isolated mesenchymal progenitor cells can be delivered separately. The delivery may be simultaneous or sequential.

In one embodiment, the endothelial progenitor cells are derived from a source including, for example, bone marrow, cord blood, peripheral blood and blood vessel walls.

In one embodiment, the mesenchymal progenitor cells are derived from a source including, for example, amniotic fluid, bone marrow, cord blood, peripheral blood and adipose tissue.

In one embodiment, the isolated and expanded endothelial progenitor cells and mesenchymal progenitor cells are cryopreserved until needed. In one embodiment, the isolated endothelial progenitor cells and mesenchymal progenitor cells are cryopreserved until needed. In one embodiment, the isolated and expanded endothelial progenitor cells and mesenchymal progenitor cells from donors can be stored in a cell bank. Important information of the donors such as gender, blood group, and HLA types are recorded for matching with future recipients. In another embodiment, the thawed progenitors cells can be further expanded prior to use.

In one embodiment, the endothelial progenitor cells and mesenchymal progenitor cells are autologous to a recipient. Endothelial progenitor cells and mesenchymal progenitor cells are isolated from a sample of peripheral blood of a patient and expanded in vitro. The same autologous endothelial progenitor cells and mesenchymal progenitor cells are then used in tissue engineered constructs which are then implanted into the same donor patient. In another embodiment, the same endothelial and mesenchymal progenitor cells are used in tissue repair and/or wound healing in the donor patient. This greatly reduces the immune rejection of the engineered tissue and implanted progenitor cells, and further eliminates the need for life-long immune suppression therapy.

In another embodiment, the endothelial progenitor cells and mesenchymal progenitor cells are not autologous to a recipient. Instead, the cells are HLA type matched to a recipient. A minimum of four matched out of the six standard HLA type-matched allele is required for there to be a match between donor and recipient.

In one embodiment, both endothelial progenitor cells and mesenchymal progenitor cells are obtained from the same source, for example, a single sample of peripheral blood. In another embodiment, the endothelial progenitor cells and mesenchymal progenitor cells are obtained from different sources, such as bone marrow or peripheral blood, for example.

In a preferred embodiment, at least endothelial progenitor cells and mesenchymal progenitor cells are present at the site where neovascularization is desired. This is accomplished by mixing the endothelial progenitor cells and mesenchymal progenitor cells to form a composition comprising enriched progenitors cells. The composition is then delivered to a TE construct, a tissue in need of repair, or wound in need of healing. In one embodiment, the method of the invention uses an enriched population of endothelial progenitor cells that is at least 10% but not more than 90% of the composition. In one embodiment, the method of the invention uses an enriched population of mesenchymal progenitor cells that is at least 10% but not more than 90% of the composition. In a preferred embodiment, the endothelial progenitor cells is 40% of the composition.

Alternately, endothelial progenitor cells and mesenchymal progenitor cells are delivered separately to a TE construct, a tissue in need of repair, wound in need of healing or a vicinity surrounding a wound in need of healing such that both progenitor cells are present at the site where neovascularization is needed. Each progenitor cells can be delivered to the same injection sites or the second progenitor cells can be delivered to an injection site adjacent to the injection site of the first progenitor cell. Adjacent sites should be close enough to each other for molecules such as growth factors to spread by passive diffusion from one site to an adjacent site, and for cells injected from one injection site to migrate to an adjacent injection site.

In one embodiment, the method of the invention comprise simultaneous delivery of an enriched populations of isolated endothelial progenitor cells and isolated mesenchymal progenitor cells to a tissue or a surrounding vicinity of a tissue in need of neovascularization. In another embodiment, an enriched populations of isolated endothelial progenitor cells and isolated mesenchymal progenitor cells are delivered sequentially to the tissue or a surrounding vicinity of a tissue in need of neovascularization.

In one embodiment, the invention provides a composition for promoting neovascularization comprising: an enriched population of isolated endothelial progenitor cells; an enriched population of isolated mesenchymal progenitor cells; and a pharmaceutically acceptable carrier. In another embodiment, the composition further comprising an extracellular matrix.

In one embodiment, the endothelial progenitor cells comprise at least 10% but not more than 90% of the total cells in the composition. In one embodiment, the mesenchymal progenitor cells comprise at least 10% but not more than 90% of the total cells in the composition. In a preferred embodiment, the endothelial progenitor cells comprise 40% and the mesenchymal progenitor cells comprise 60% of the total cells of the composition.

In one embodiment, the invention provides a kit comprising: an enriched population of isolated endothelial progenitor cells; and an enriched population of isolated mesenchymal progenitor cells. In another embodiment, the kit further comprising an extracellular matrix or a biocompatible scaffold. In another embodiment, the kit comprise instructions on the use of the components in the kit, for example, mixing the populations of progenitor cells for direct injection into a tissue in need of repair or wound healing, or for neovascularization of tissue engineered constructs. In another embodiment, each kit comprises the donor's information such as gender, blood group and the six standard HLA type that is known in the art.

### BRIEF DESCRIPTION OF THE DRAWINGS

FIG. 1A. CD31-selected cbEPCs were evaluated at passage 6. HDMECs and HSVSMCs served as positive and negative controls respectively. Cytometric analysis of cultured cbEPCs for endothelial markers CD34, VEGF-R2, CD146, CD31, vWF and CD105, the mesenchymal marker CD90, and hematopoietic/monocytic markers CD45 and CD14. Solid gray histograms represent cells stained with fluorescent antibodies. Isotype-matched controls are overlaid in a black line on each histogram.

FIG. 1B. Up-regulation of E-selectin, ICAM-1 and VCAM-1 in cultured cbEPC in response to TNF-α. Solid gray histograms represent cells stained with fluorescent antibodies while black lines correspond to the isotype-matched control fluorescent antibodies.

FIG. 2A. In vitro expansion of cbEPCs and adult blood EPCs isolated from mononuclear cells and purified by CD31-positive selection.

FIG. 2B. Growth curves of cbEPCs at different passage numbers (P4, P6, P9, P12 and P15). Each data point represents the mean of three separate cultures ±SD.

FIG. 2C. Doubling time profiles of cbEPCs at different passage numbers. Values were calculated from the mean values of cell number obtained at specific time points after plating.

FIG. 2D. Morphological differences of cbEPCs at increasing passage. Each bar represents the mean area ±SD obtained from randomly selected fields. All values were normalized to the total cell area occupied by HDMECs. *P<0.05 compared to HDMECs.

FIG. 3. Proliferative response toward angiogenic factors of cbEPCs at different passage numbers (P4, P6, P9, P12 and P15). Each bar represents the mean of three separate cultures ±SD, with values normalized to the values of cell density obtained at 24 hours when treatment began. *P<0.05 compared to control. ‡P<0.05 compared to equivalent treatment on HDMECs.

FIG. 4. Microvessel density in Matrigel implants was quantified by counting lumenal constructs containing red blood cells. Each bar represents the mean microvessel density value determined from four separated implants and animals ±SD. * P<0.05 compared to HDMEC. †P<0.05 compared to cbEPC-P3.‡P<0.05 compared to cbEPC-P6.

FIG. 5. Microvessel density was quantified by counting lumenal structures containing red blood cells. Each bar represents the mean microvessel density value determined from four separated implants and animals ±SD. *P<0.05 compared to x1/3.†P<0.05 compared to x1.

FIG. 6A. Morphology of cbEPCs (cobblestone), bMPCs and cbMPCs (spindle) in culture (scale bars, 100 μm).

FIG. 6B. cbEPCs and MPCs were serially passaged and their in vitro expansion potential estimated by the accumulative cell numbers obtained from 25 mL of either cord blood or bone marrow samples after 25, 40 and 60 days in culture.

FIG. 6C. Flow cytometric analysis of cbEPCs, bmMPCs and cbMPCs. Solid gray histograms represent cells stained with fluorescent antibodies. Isotype-matched controls are overlaid in a black line on each histogram.

FIG. 7. PDGF-Rβ expression on MPCs. Histogram of PDGF-Rβ expression bmMPCs and cbMPCs in culture. PDGF-Rβ expression was up-regulated by TGF-β1 and down-regulated by PDFG-BB. SMCs obtained from human sapheneous vein served as control.

FIG. 8A and B. Macroscopic view of explanted Matrigel plugs seeded with 40% cbEPCs:60% bmMPCs.

FIG. 8C. Macroscopic view of explanted Matrigel plugs seeded with 40% cbEPCs:60% cbMPCs.

FIG. 8D. Microvessel density of implants with various ratios of cbEPCs:MPCs; n≧4 each condition). Each bar represents the mean ±S.D. (vessels/mm²) obtained from only vascularized implants. *P<0.05 compared to implants with bmMPCs alone (n=4). †P<0.05 compared to implants with cbMPCs alone (n=4).

FIG. 9. In vitro secretion of VEGF. Quantitative measurement of human VEGF in the cell culture supernatant of bmMPCs and cbMPCs. VEGF values were normalized to total cell number determined at the time of supernatant collection.

FIG. 10. Quantification of microvessel density was performed by counting erythrocyte-filled vessels. Each bar represents the mean microvessel density value determined from four separate implants and mice ±S.D. (vessels/mm²).

FIG. 11. Cellularity stabilization on the Matrigel implants containing 40% cbEPCs and 60% bmMPCs at various days post-implantation. Values reported correspond to the average cellularity expressed as cells/mm² ±S.D. *P<0.05 compared to implants at day 7 (n=4).

FIG. 12. Histogram of microvessel density in implants seeded with bmMPCs in the absence or presence of either abEPCs or cbEPCs. Each bar represents the mean microvessel density determined ±S.D. (vessels/mm²). *P<0.05 compared to implants with bmMPCs alone (n=4).

### DETAILED DESCRIPTION OF THE INVENTION

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean ±1%.

The creation of vascular networks is crucial for the success of therapeutic neovascularization in regenerative medicine such as tissue-engineered (TE) organs and tissues, in the recovery of ischemic organs and tissues, and also for wound healing. To guarantee an appropriate provision of nutrients, gas exchange, and elimination of waste products, engineered tissues must have the capacity to generate a vascular network that anastomoses with the host vasculature shortly after implantation. Increased blood flow via new vascular network can speed recovery and healing in ischemic organs and tissues, and in chronic wounds. Currently, there are no TE constructs clinically available with an inherent microvascular bed, and therefore successes in TE have been restricted to the replacement of relatively thin (skin) or avascular tissues (cartilage), where post-implantation neovascularization from the host is sufficient.

To overcome this problem of neovascularization, several therapeutic strategies have been proposed and tested. These strategies center on promoting angiogenesis—ingrowth of microvessels by delivering angiogenic molecules such as VEGF, either as proteins or via gene transfer to the tissues needing neovascularization or re-neovascularization. However, these strategies cannot provide rapid and complete neovascularization of thick tissues, engineered or natural. A complete neovascularization of tissues, whether the tissues are engineered tissue or naturally existing tissue in an organism, requires the additional process of vasculogenesis.

In vivo vasculogenesis can be promoted by exploiting the inherent vasculogenic ability of endothelial cells (ECs). Earlier studies using human umbilical vein ECs (HUVECs) and human microvascular ECs (HDMECs) showed the feasibility of engineering microvascular networks in vivo (Koike, N., et. al., 2004, Nature, 428:138-9; Nor, J. E., et. al., 2001, Lab. Invest. 81:453-63; Schechner, J. S. et. al., 2000, Proc. Natl. Acad. Sci. USA, 97: 9191-6). However, the clinical use of mature ECs derived from autologous vascular tissue is limited by the difficulty of obtaining sufficient quantities of cells with minimal donor site morbidity. In addition, the studies by Schechner and Nor required genetic modification of the mature EC using the anti-apoptotic gene bc1-2, which could participate in alteration of the cells to a cancerous state.

The present invention relates to using at least two types of cells, endothelial progenitor cell (EPC) and mesenchymal progenitor cell (MPC), for the neovascularization of TE constructs and in therapeutic neovascularization useful in treating ischemic tissues and in wound healing. Populations of these progenitor cells are isolated from sources such as circulating peripheral blood, umbilical cord blood, bone marrow, and adipose tissue. The isolated population of progenitor cells are then enriched by various methods known in the art and expanded through multiple cell divisions to produce sufficient number of progenitor cells for the methods of the invention disclosed herein.

There are several advantages to using EPCs and MPCs for vascular network formation. Progenitor cells are immature or undifferentiated cells, and they have greater cell division capability. Therefore, it is possible to culture in vitro the desired progenitor cells to obtain sufficient quantities for the neovascularization of engineered tissues and in therapeutic vasculogenesis. Moreover both EPCs and MPCs are present in the circulating blood and can be isolated from a single sample of blood, for example, circulating peripheral blood. The isolated EPCs and MPCs can then be expanded in vitro prior to use. Accordingly, it is possible to obtain autologous EPCs and MPCs from a patient for the neovascularization of a engineered tissue which will be implanted back into the same patient. Autologous EPCs and MPCs can be used for neovascularization of ischemic tissues and organs, and for chronic wounds. This greatly reduces the problem of tissue rejection in recipients of engineered tissues or immune response rejecting the progenitor cells that are implanted into ischemic organs and tissues, and in chronic wounds. Examples of organs and tissues that can become ischemic and treated using the invention disclosed herein include but are not limited to the heart, muscles, skin, adipose tissue, brain, bone, liver, lungs, intestines, legs, limbs, and kidneys.

In one embodiment, the autologous EPCs and MPCs can be used for the invention disclosed herein. Prior to major surgery to repair certain defects, a patient can donate a sample of human bone marrow or peripheral blood for the isolation and expansion of EPCs and MPCs. In another embodiment, if a patient suffered from a chronic wound that is slow in healing naturally or if the patient had recently suffered a heart attack or stroke, the patient can donate a sample of his or her own human bone marrow or peripheral blood for the purpose of isolation and expansion of EPCs and MPCs. In another embodiment, EPCs and MPCs can be isolated for the purpose of pre-banking the progenitor cells in high risk populations, for example those serving in the military. In the event that a solder is injured and left missing a part of or a whole organ, tissue, and/or body parts such as facial bones, the solder's previously banked EPCs and MPCs can be utilized for TE projects to reconstruct the missing organ, tissue, and/or body parts. The EPCs and MPCs can also be useful in speeding healing and recovery of the solder's injuries. Enriched populations of EPCs and MPCs are obtained from the isolated and expanded EPCs and MPCs respectively from suitable sources disclosed herein. A composition comprising of an enriched population of isolated autologous EPCs and an enriched population of isolated autologous MPCs can used in TE constructs which will be later implanted in the patient. In another aspect, the composition can be injected directly to the wound to aid healing, to the tissue to speed up tissue repair, and/or to the healthy tissue adjacent the wound or ischemic part of the heart or other ischemic organs and tissues in the body.

In one embodiment, the EPCs and MPCs are human leukocyte antigen (HLA) typed matched for the recipient of the cells. In one embodiment, EPCs and MPCs are isolated and expanded from a single donor and the progenitor cells are matched for at least 4 out of 6 alleles of the HLA class I: HLA-A and HLA-B; and HLA class II: DRB1 with the recipient. In another embodiment, EPCs and MPCs are isolated and expanded from different donors and the progenitor cells are HLA type matched for at least 4 out of 6 alleles of the HLA class I: HLA-A and HLA-B; and HLA class II: DRB1 with the recipient.

Envisioned in the invention is a bank of cells which comprises a composition comprising an enriched population of isolated EPCs and an enriched population of isolated MPCs. In one embodiment, the bank of cells comprises a composition comprising an enriched population of isolated EPCs. In another embodiment, the bank of cells comprises a composition comprising an enriched population of isolated MPCs. In one embodiment, the progenitor cells are isolated in vitro and then cryopreserved for the bank of cells. In one embodiment, the progenitor cells are isolated and expanded in vitro prior to cryopreservation for the bank of cells. When EPCs and MPCs are need for any neovascularization, the cryopreserved EPCs and MPCs of the cell bank can be utilized.

In one embodiment, the recipient of a composition comprising an enriched population of isolated EPCs and an enriched population of isolated MPCs is a mammal. Examples of mammals include but are not limited to dog, cat, sheep, goat, monkeys, pigs and human. In a preferred embodiment, the recipient is a human.

Embodiments of the invention provides a method of promoting neovascularization in a tissue in need thereof comprising contacting the tissue with a composition comprising an enriched population of isolated EPCs and an enriched population of isolated MPCs, wherein the EPCs and MPCs induce the formation of new blood vessels with functional connections to the host vasculature. Tissues in need of neovascularization include all TE constructs that are greater than 2 mm in thickness and are tissues that are normally vascularized in the human body. For example, tissue engineered heart valves, cardiac muscles, bladder, pancreas, and liver to name a few. The neovascularization of such engineered tissues, when implanted into a mammal, ensures the survival and functionality of the tissue in the mammalian host. In accordance with the invention disclosed herein, the presence of EPCs and MPCs in the TE construct enables the tissue to form de novo blood vessels that anastamose with the existing host circulatory network at the site of implantation. Formation of an adequate vascular network will provide a constant supply of oxygen and nutrients for the engineered tissue as well as facilitate efficient removal of toxic metabolic waste products. A constant supply of oxygen and nutrients is necessary for the engineered tissue to grow, remodel, and perform its biological function in the body.

During the process of neovascularization, both EPCs and MPCs work together to form de novo blood vessels. New branched of blood vessels form from existing blood vessels, and they join up with the de novo vessels to form a network. The EPCs mature and differentiate into ECs which forms the tunica intima—thinnest and inner walls of the blood vessels; the MPCs give rise to smooth muscle cells that make up the bulk of the tunica media—the thickest layer and tunica adventitia—connective tissue layer of a blood vessel.

It is envisioned that the genome of the isolated EPCs and isolated MPCs can include additional gene-encoding DNA, for example, the coding gene for the green fluorescent protein, an enzyme, a growth factor or cytokine. The extra protein, when expressed, can be used to track the migration and differentiation of the progenitor cells. The extra enzyme, growth factor and/or cytokine can be used to replenished local deficiencies that have resulted from disease or genetic defects. The extra gene-encoding DNA can be introduced into the genome by transfection methods known to one skilled in the art, such as electroporation and lipid-based Lipofectamine transfection.

Unless otherwise stated, the present invention was performed using standard procedures and methods known in the art for tissue culture and tissue engineering, as described, for example, in Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

### DEFINITIONS

As used herein, the term “angiogenesis” refers to the formation of new blood vessels from pre-existing blood vessels.

As used herein, the term “vasculogenesis” refers to the formation of new blood vessels when there are no pre-existing ones. Blood vessel formation occurring by a de novo process where EPCs and MPCs migrate, assemble and differentiate in response to local cues (such as growth factors and extracellular matrices) to form new blood vessels.

As used herein, “neovascularization” refers to the formation of functional vascular networks that may be perfused by blood or blood components. Neovascularization includes angiogenesis, budding angiogenesis, intussuceptive angiogenesis, sprouting angiogenesis, therapeutic angiogenesis and vasculogenesis. Therapeutic neovascularization refers to the formation of vascular network in ischemic tissues, wound, and adjacent tissue around the wound.

As used herein, the term “adjacent” refers to close enough to a wound for molecules such as growth factors to spread by passive diffusion from the adjacent tissue to the wound, and for cells injected at the adjacent tissue to migrate to the wound site.

As used herein, the term “progenitor” cell refers to an immature or undifferentiated cell, typically found in post-natal animals. Progenitor cells can be unipotent or multipotent. As used herein, progenitor cells refers to either EPCs or MPCs, or both EPCs and MPCs.

As used herein, the term “autologous” refers to a situation in which the donor of the progenitor cells and recipient of the progenitor cells and/or engineered tissue are the same person.

The term “isolated” as used herein signifies that the cells are placed into conditions other than their natural environment. The term “isolated” does not preclude the later use of these cells thereafter in combinations or mixtures with other cells.

As used herein, the term “expanding” refers to increasing the number of like cells through cell division (mitosis). The term “proliferating” and “expanding” are used interchangeably.

As used herein, “cryopreservation” refers to the preservation of cells by cooling to low sub-zero temperatures, such as (typically) 77 K or −196° C. (the boiling point of liquid nitrogen). Cryopreservation also refers to storing the cells at a temperature between 0-10° C. in the absence of any cryopreservative agents. At these low temperatures, any biological activity, including the biochemical reactions that would lead to cell death, is effectively stopped. Cryoprotective agents are often used at sub-zero temperatures to preserved the cells from damaged due to freezing at low temperatures or warming to room temperature.

As used herein, “composition” refers to an injectate, substance or a combination of substances which can be delivered into a tissue, an organ, or a tissue engineered construct such a gel-like extracellular matrix or a biocompatible scaffold, and are used interchangeably herein. Exemplary compositions include, but are not limited to, a suspension of progenitor cells in a suitable physiologic carrier such as saline.

As used here, “delivery” refers to providing a composition to a treatment site in an injured tissue through any method appropriate to deliver the functional composition to the treatment site; or deliver to a TE construct such as a biocompatible scaffold. Non-limiting examples of delivery methods include direct injection at the treatment site, direct topical application at the treatment site, percutaneous delivery for injection, percutaneous delivery for topical application, and other delivery methods well known to persons of ordinary skill in the art.

As used herein, “ischemic” refers to the reduced or elimination of blood flow in a tissue or organ such that the tissue or organ is deprived of oxygen. The tissue or organ experiences hypoxia. This happens generally due to factors in the blood vessels, such blocked blood vessels or rupture blood vessels, with resultant damage or dysfunction of ischemic tissue or organ. Tissues include, for example, the heart, skin, adipose tissue, muscle, brain, bone, liver, lungs, intestines, the limbs and kidneys. Ischemic diseases that lead to ischemic tissues include, for example, cerebrovascular ischemia, renal ischemia, pulmonary ischemia, limb ischemia, ischemic cardiomyopathy and myocardial ischemia.

As used herein, the terms “tissue regeneration”, “tissue engineering” and “regenerative medicine” are related terms and used interchangeably.

As used herein, the word “repair”, means the natural replacement of worn, torn or broken components with newly synthesized components. The word “healing”, as used herein, means the returning of torn and broken organs and tissues (wounds) to wholeness.

As used herein, the term “tissue engineered construct” or TE construct” or construct refers to a product made by assembling adherent cells on to a scaffold using the techniques of tissue engineering that is known in the art.

As used herein, the term “biocompatible” refers to the ability to replace part of a living system or to function in intimate contact with living tissue. A biocompatible material is a synthetic or natural material used to replace part of a living system or to function in intimate contact with living tissue. Biocompatible materials are intended to interface with biological systems to evaluate, treat, augment or replace any tissue, organ or function of the body.

### Endothelial Progenitor Cells (EPC)

EPCs are primitive cells thought to originate in the bone marrow or derived from the blood vessel walls. EPCs are released into the bloodstream. These circulating, bone marrow-derived EPCs go to areas of blood vessel injury to help repair the damage. They have the ability to expand and differentiate into ECs, the cells that make up the inner lining of blood vessels, and are known to participate in both vasculogenesis and vascular homeostasis.

Sources of EPCs include human umbilical cord blood, human bone marrow, human circulating peripheral blood, and blood vessel walls. In one embodiment, EPCs of the invention can be isolated from circulating peripheral blood and the umbilical cord blood. From a sample of blood, the mononuclear cell fraction (MNC) of the blood is obtained by percoll gradient centrifugation. This MNC fraction can be further purified for EPCs based on the CD34/CD133+ surface markers of EPCs and then expanded in culture using EPC medium. EPC medium: EGM-2 (Endothelial Basal Medium (EBM-2)+SingleQuots; hydrocortisoneis excluded; Lonza, Walkersville, Md.), 20% fetal bovine serum (FBS) and 1× glutamine-penicillin-streptomycin (GPS; Invitrogen, Carlsbad, Calif.). In one embodiment, human serum, either autologous or allogeneic AB serum, or human platelet rich plasma supplemented with heparin (2 U/ml) can be used instead of FBS. Alternatively, the MNC fraction can be grown in tissue culture directly. Non-adherent cells are removed 48 hours later (for cord blood) and 4 days later (for periphery blood). After being in culture for 2-3 weeks, the cells are confluent and are then selected for CD31, another surface marker of EPCs. At this time the EPCs have a cobblestone-like morphology in culture, positive for the following markers: CD34, KDR, CD146, CD31, CD105, VE-cadherin, vWF, and eNOS; and negative for CD90, CD45, and CD14. In addition the EPCs response to the TNF-α by up regulating expression of E-selectin, ICAM-I and VCAM-1. Over the course of the next 1-7 weeks in culture, the EPCs expand exponential with 30-70 cells population doublings. The EPCs and EC specific markers can be monitored by methods known in the art, for example, flow cytometry using specific antibodies against the various cell surface markers. A population enriched in isolated EPCs is at least 90% positive for CD31 and VE-cadherin, and no more than 5% positive for CD90, CD45, and CD14.

Other methods of isolating, culture and expansion of EPCs are described by Jonathan M. Hill, 2003, NEJM, 348:593-600; Eggermann J, 2003, Cardiovasc Res., 58(2):478-86; Hristov, et al., 2003, Trends in Cardiovascular Medicine 13 (5): 201-6; Amelia Casamassimi et. al, 2007, J. Biochemistry, 141:503-11; and U.S. Pat. No. 5,980,887, and U.S. Patent application Nos. 20030194802, 20060035290, and 2006010385 and are hereby incorporated by reference.

### Mesenchymal Progenitor Cells (MPC)

MPCs are cells derived from the mesoderm and they have a large capacity for self-renewal while maintaining their multipotency. MPCs are undifferentiated mesenchymal cells that are capable of expanding and differentiating into more than one specific type of mesenchymal tissue cells. Cell types that MPCs have been shown to differentiate into in vitro or in vivo include osteoblasts, chondrocytes, myocytes, and adipocytes. MPCs are also referred to as mesenchymal stem cells (MSC) and they are used herein interchangeably.

Sources of MPCs include human amniotic fluid, human bone marrow, human umbilical cord blood, human circulating peripheral blood, and human adipose tissue. MPCs are isolated, for example, from the mononuclear cell fraction of umbilical cord blood or peripheral blood. The MNC fraction is grown in MPC culture media: EGM-2 (Endothelial Basal Medium (EBM-2)+SingleQuots; VEGF, bFGF, hydrocortisone, heparin are excluded; Lonza, Walkersville, Md.), 20% fetal bovine serum (FBS) and 1× GPS (Invitrogen, Carlsbad, Calif.). In one embodiment, human serum, either autologous or allogeneic AB serum, or human platelet rich plasma supplemented with heparin (2 U/ml) can be used instead of FBS. At this stage these MPCs have a mesenchymal-like morphology (spindle-like) and express specific mesenchymal cell markers (positive for CD90, α-SMA, Calponin, CD44, CD105, CD29 and CD146) and do not express hematopoietic (negative for CD14 and CD45) and endothelial cell markers (negative for CD31, VE-Cadherin and vWF) (Pitting et. al., 1999, Science 284:143-147; Kaviani et. al., 2001, J. Pediatr. Surg. 36: 1662-5; Kunisaki et. al., 2007, J. Pediatr. Surg. 42:974-9). A population enriched in MPCs is at least 90% positive for CD90, and no more than 5% positive for CD45, and CD31.

Other methods of isolation and expansion of MPCs are described in Current Protocols in Stem Cell Biology 2007 (Steigman, S. A. and Fauza, D. O.) (Mick Bhatia, et. al., ed., John Wiley and Sons, Inc.) and in U.S. Pat. Nos. 5,486,359, 6,387,367, 7,060,494) and are hereby incorporated by reference.

In one embodiment, the isolated EPCs and MPCs are autologous to a recipient.

In another embodiment, a single sample of peripheral blood can be used for isolating and expanding the EPCs and MPCs. After isolation and expansion, the EPCs and MPCs can be cryopreserved by methods known in the art. In one embodiment, the isolated EPCs and MPCs can be cryopreserved by methods known in the art.

### Cryopreservation of Cells

In one embodiment, the invention provides a cryopreserved composition comprising an enriched population of isolated EPCs and an enriched population of isolated MPCs; an amount of cryopreservative sufficient for the cryopreservation of the isolated progenitor cells; and a pharmaceutically acceptable carrier. In one embodiment, the cryopreserved composition comprises a composition comprising an enriched population of isolated EPCs; an amount of cryopreservative sufficient for the cryopreservation of the isolated EPCs; and a pharmaceutically acceptable carrier. In another embodiment, the cryopreserved composition comprises a composition comprising an enriched population of isolated MPCs; an amount of cryopreservative sufficient for the cryopreservation of the isolated MPCs; and a pharmaceutically acceptable carrier.

Freezing is destructive to most living cells. Upon cooling, as the external medium freezes, cells equilibrate by losing water, thus increasing intracellular solute concentration. Below about 10°-15° C., intracellular freezing will occur. Both intracellular freezing and solution effects are responsible for cell injury (Mazur, P., 1970, Science 168:939-949). It has been proposed that freezing destruction from extracellular ice is essentially a plasma membrane injury resulting from osmotic dehydration of the cell (Meryman, H. T., et al., 1977, Cryobiology 14:287-302).

Cryoprotective agents and optimal cooling rates can protect against cell injury. Cryoprotection by solute addition is thought to occur by two potential mechanisms: colligatively, by penetration into the cell, reducing the amount of ice formed; or kinetically, by decreasing the rate of water flow out of the cell in response to a decreased vapor pressure of external ice (Meryman, H. T., et al., 1977, Cryobiology 14:287-302). Different optimal cooling rates have been described for different cells. Various groups have looked at the effect of cooling velocity or cryopreservatives upon the survival or transplantation efficiency of frozen bone marrow cells or red blood cells (Lovelock, J. E. and Bishop, M. W. H., 1959, Nature 183:1394-1395; Ashwood-Smith, M. J., 1961, Nature 190:1204-1205; Rowe, A. W. and Rinfret, A. P., 1962, Blood 20:636; Rowe, A. W. and Fellig, J., 1962, Fed. Proc. 21:157; Rowe, A. W., 1966, Cryobiology 3(1):12-18; Lewis, J. P., et al., 1967, Transfusion 7(1):17-32; Rapatz, G., et al., 1968, Cryobiology 5(1):18-25; Mazur, P., 1970, Science 168:939-949; Mazur, P., 1977, Cryobiology 14:251-272; Rowe, A. W. and Lenny, L. L., 1983, Cryobiology 20:717; Stiff, P. J., et al., 1983, Cryobiology 20:17-24; Gorin, N. C., 1986, Clinics in Haematology 15(1):19-48).

The successful recovery of human bone marrow cells after long-term storage in liquid nitrogen has been described (1983, American Type Culture Collection, Quarterly Newsletter 3(4): 1). In addition, stem cells in bone marrow were shown capable of withstanding cryopreservation and thawing without significant cell death, as demonstrated by the ability to form equal numbers of mixed myeloid-erythroid colonies in vitro both before and after freezing (Fabian, I., et al., 1982, Exp. Hematol 10:119-122). The cryopreservation and thawing of human fetal liver cells (Zuckerman, A. J., et al., 1968, J. Clin. Pathol. (London) 21(1):109-110), fetal myocardial cells (Robinson, D. M. and Simpson, J. F., 1971, In Vitro 6(5):378), neonatal rat heart cells (Alink, G. M., et al., 1976, Cryobiology 13:295-304), and fetal rat pancreases (Kemp, J. A., et al., 1978, Transplantation 26(4):260-264) have also been reported.

The injurious effects associated with freezing can be circumvented by (a) use of a cryoprotective agent, (b) control of the freezing rate, and (c) storage at a temperature sufficiently low to minimize degradative reactions.

Cryoprotective agents which can be used include but are not limited to dimethyl sulfoxide (DMSO) (Lovelock, J. E. and Bishop, M. W. H., 1959, Nature 183:1394-1395; Ashwood-Smith, M. J., 1961, Nature 190:1204-1205), glycerol, polyvinylpyrrolidine (Rinfret, A. P., 1960, Ann. N.Y. Acad. Sci. 85:576), polyethylene glycol (Sloviter, H. A. and Ravdin, R. G., 1962, Nature 196:548), albumin, dextran, sucrose, ethylene glycol, i-erythritol, D-Sorbitol, D-mannitol (Rowe, A. W., et al., 1962, Fed. Proc. 21:157), D-sorbitol, i-inositol, D-lactose, choline chloride (Bender, M. A., et al., 1960, J. Appl. Physiol. 15:520), amino acids (Phan The Tran and Bender, M. A., 1960, Exp. Cell Res. 20:651), methanol, acetamide, glycerol monoacetate (Lovelock, J. E., 1954, Biochem. J. 56:265), and inorganic salts (Phan The Tran and Bender, M. A., 1960, Proc. Soc. Exp. Biol. Med. 104:388; Phan The Tran and Bender, M. A., 1961, in Radiobiology, Proceedings of the Third Australian Conference on Radiobiology, Ilbery, P. L. T., ed., Butterworth, London, p. 59). In a preferred embodiment, DMSO is used, a liquid which is non-toxic to cells in low concentration. Being a small molecule, DMSO freely permeates the cell and protects intracellular organelles by combining with water to modify its freezability and prevent damage from ice formation. Addition of plasma (e.g., to a concentration of 20-25%) can augment the protective effect of DMSO. After addition of DMSO, cells should be kept at 0-4° C. until freezing, since DMSO concentrations of about 1% are toxic at temperatures above 4° C.

A controlled slow cooling rate is critical. Different cryoprotective agents (Rapatz, G., et al., 1968, Cryobiology 5(1):18-25) and different cell types have different optimal cooling rates (see e.g., Rowe, A. W. and Rinfret, A. P., 1962, Blood 20:636; Rowe, A. W., 1966, Cryobiology 3(1):12-18; Lewis, J. P., et al., 1967, Transfusion 7(1):17-32; and Mazur, P., 1970, Science 168:939-949 for effects of cooling velocity on survival of marrow-stem cells and on their transplantation potential). The heat of fusion phase where water turns to ice should be minimal. The cooling procedure can be carried out by use of, e.g., a programmable freezing device or a methanol bath procedure.

Programmable freezing apparatuses allow determination of optimal cooling rates and facilitate standard reproducible cooling. Programmable controlled-rate freezers such as Cryomed or Planar permit tuning of the freezing regimen to the desired cooling rate curve. For example, for marrow cells in 10% DMSO and 20% plasma, the optimal rate is 1 to 3° C./minute from 0° C. to −80° C. The container holding the cells must be stable at cryogenic temperatures and allow for rapid heat transfer for effective control of both freezing and thawing. Sealed plastic vials (e.g., Nunc, Wheaton Cryules®) or glass ampules can be used for multiple small amounts (1-2 ml), while larger volumes (100-200 ml) can be frozen in polyolefin bags (e.g., Delmed) held between metal plates for better heat transfer during cooling. (Bags of bone marrow cells have been successfully frozen by placing them in −80° C. freezers which, fortuitously, gives a cooling rate of approximately 3° C./minute).

In an alternative embodiment, the methanol bath method of cooling can be used. The methanol bath method is well-suited to routine cryopreservation of multiple small items on a large scale. The method does not require manual control of the freezing rate nor a recorder to monitor the rate. In a preferred aspect, DMSO-treated cells are pre-cooled on ice and transferred to a tray containing chilled methanol which is placed, in turn, in a mechanical refrigerator (e.g., Harris or Revco) at −80° C. Thermocouple measurements of the methanol bath and the samples indicate the desired cooling rate of 1° to 3° C./minute. After at least two hours, the specimens have reached a temperature of −80° C. and can be placed directly into liquid nitrogen (−196° C.) for permanent storage.

After thorough freezing, cells can be rapidly transferred to a long-term cryogenic storage vessel. Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum.

In one embodiment, the cryopreservation procedure described in Current Protocols in Stem Cell Biology, 2007, (Mick Bhatia, et. al., ed., John Wiley and Sons, Inc.) is used for the compositions of isolated and expanded progenitor cells described herein and is hereby incorporated by reference. Mainly when the EPCs or MPCs on a 10-cm tissue culture plate have reached at least 50% confluency, preferably 70% confluency, the media within the plate is aspirated and the progenitor cells are rinsed with phosphate buffered saline. The adherent progenitor cells are then detached by 3 ml of 0.025% trypsin/0.04%EDTA treatment. The trypsin/EDTA is neutralized by 7 ml of media and the detached progenitor cells are collected by centrifugation at 200×g for 2 min. The supernatant is aspirated off and the pellet of progenitor cells is resuspended in 1.5 ml of media. The harvested progenitor cells are cryopreserved at a density of at least 3×10³ cells/ml. A aliquot of 1 ml of 100% DMSO is added to the suspension of progenitor cells and gently mixed. Then 1 ml aliquots of this suspension of progenitor cells in DMSO is dispensed into cyrules in preparation for cryopreservation. The sterilized storage cryules preferably have their caps threaded inside, allowing easy handling without contamination. Suitable racking systems are commercially available and can be used for cataloguing, storage, and retrieval of individual specimens.

Other methods of cryopreservation of viable cells, or modifications thereof, are available and envisioned for use (e.g., cold metal-mirror techniques; Livesey, S. A. and Linner, J. G., 1987, Nature 327:255; Linner, J. G., et al., 1986, J. Histochem. Cytochem. 34(9):1123-1135; U.S. Pat. Nos. 4,199,022, 3,753,357, 4,559,298 and are incorporated hereby reference.

Recovering Progenitor Cells from the Frozen State

When the progenitor cells are needed for vasculogenesis, such as when a tissue is being engineered or a patient has recently suffered a heart attack or stroke, the frozen EPCs and MPCs can be thawed according to methods known in the art, mixed in appropriate ratios and incorporated into the engineered tissue or ischemic tissue or organ.

Frozen progenitor cells are preferably thawed quickly (e.g., in a water bath maintained at 37°-41° C.) and chilled on ice immediately upon thawing. In particular, the cryogenic vial containing the frozen progenitor cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

In a particular embodiment, the thawing procedure after cryopreservation is described in Current Protocols in Stem Cell Biology 2007 (Mick Bhatia, et. al., ed., John Wiley and Sons, Inc.) and is hereby incorporated by reference. Immediately after removing the cryogenic vial from the cryo-freezer, the vial is rolled between the hands for 10 to 30 sec until the outside of the vial is frost free. The vial is then held upright in a 37° C. water-bath until the contents are visibly thawed. The vial is immersed in 95% ethanol or sprayed with 70% ethanol to kill microorganisms from the water-bath and air dry in a sterile hood. The contents of the vial is then transferred to a 10-cm sterile culture containing 9 ml of media using sterile techniques. The progenitor cells can then be cultured and further expanded in a incubator at 37° C. with 5% humidified CO₂.

It may be desirable to treat the progenitor cells in order to prevent cellular clumping upon thawing. To prevent clumping, various procedures can be used, including but not limited to, the addition before and/or after freezing of DNase (Spitzer, G., et al., 1980, Cancer 45:3075-3085), low molecular weight dextran and citrate, hydroxyethyl starch (Stiff, P. J., et al., 1983, Cryobiology 20:17-24).

The cryoprotective agent, if toxic in humans, should be removed prior to therapeutic use of the thawed progenitor cells. In an embodiment employing DMSO as the cryopreservative, it is preferable to omit this step in order to avoid cell loss, since DMSO has no serious toxicity. However, where removal of the cryoprotective agent is desired, the removal is preferably accomplished upon thawing.

One way in which to remove the cryoprotective agent is by dilution to an insignificant concentration. This can be accomplished by addition of medium, followed by, if necessary, one or more cycles of centrifugation to pellet the cells, removal of the supernatant, and resuspension of the cells. For example, the intracellular DMSO in the thawed cells can be reduced to a level (less than 1%) that will not adversely affect the recovered cells. This is preferably done slowly to minimize potentially damaging osmotic gradients that occur during DMSO removal.

After removal of the cryoprotective agent, cell count (e.g., by use of a hemocytometer) and viability testing (e.g., by trypan blue exclusion; Kuchler, R. J. 1977, Biochemical Methods in Cell Culture and Virology, Dowden, Hutchinson & Ross, Stroudsburg, Pa., pp. 18-19; 1964, Methods in Medical Research, Eisen, H. N., et al., eds., Vol. 10, Year Book Medical Publishers, Inc., Chicago, pp. 39-47) can be done to confirm cell survival.

Other procedures which can be used, relating to processing of the thawed cells, include enrichment for adherent progenitor cells and expansion by in vitro culture as described supra.

In a preferred, but not required, aspect of the invention, thawed cells are tested by standard assays of viability (e.g., trypan blue exclusion) and of microbial sterility as described herein, and tested to confirm and/or determine their identity relative to the recipient.

Endotoxin levels can be determined by the gel-clot limulus amebocyte lysate (LAL) test method in compliance with the US Food and Drug Administration's GMP regulations, 21 CFR §211. Acceptable endotoxin level is 5.0 EU/ml.

An aliquot of the cells will be taken prior to cryopreservation for mycoplasma PCR testing. The Mycoplasma PCR testing will be performed at a GMP approved facility using MycoSensor™ QPCR Assay Kit (Manufactured by Stratagene).

Methods for identity testing which can be used include but are not limited to HLA typing (Bodmer, W., 1973, in Manual of Tissue Typing Techniques, Ray, J. G., et al., eds., DHEW Publication No. (NIH) 74-545, pp. 24-27), and DNA fingerprinting, which can be used to establish the genetic identity of the cells. DNA fingerprinting (Jeffreys, A. J., et al., 1985, Nature 314:67-73) exploits the extensive restriction fragment length polymorphism associated with hypervariable minisatellite regions of human DNA, to enable identification of the origin of a DNA sample, specific to each individual (Jeffreys, A. J., et al., 1985, Nature 316:76; Gill, P., et al., 1985, Nature 318:577; Vassart, G., et al., 1987, Science 235:683), and is thus preferred for use.

### Formation of Functional Anastomoses

Neovascularization can be created in vivo using EPCs and MPCs isolated and purified from umbilical blood cord, periphery blood, or bone marrow. In example 1, implanted Matrigel xenographs containing 4:1 ratio of EPCs to MPCs exhibited the presence of murine red blood cells-containing blood vessels seven days post-implantation. This indicates the formation of functional anastomoses with the murine circulatory system of the host. Therefore microvascular networks can be created within a tissue using human autologous EPCs and MPCs obtained from umbilical cord blood, periphery blood, or bone marrow. This invention could be applied widely to any tissue-engineered organ or tissue that requires a blood supply, and even any tissue in the body that is ischemic as a result of illness and diseases such as congestive heart failure, poor circulation, obesity, lymphatic obstructions and diabetes.

In one embodiment, the EPCs and MPCs are mixed together to achieve microneovascularization in vivo. Just EPCs alone or just MPCs alone do not promote microneovascularization in vivo in the absence of the other cell type. The cell composition of EPC and MPC comprises at least 10% of each cell type. In one embodiment, the percentage of EPC in the composition is about 10%, 20%, 30%, 40%, 50%, 60%, 70%, 80%, 90%, and all the percentages between 10-90%. In one embodiment, the percentage of MPC in the composition is about 10%, 20%, 30%, 40%, 50%, 60%, 70%, 80%, 90%, and all the percentages between 10-90%. The EPC and MPC are mixed to obtain a final 100%. In a preferred embodiment, the percentage ratio of EPC to MPC is 40%: 60%.

In one embodiment, the EPCs are capable of differentiating into ECs and forming small blood vessel in the presence of smooth muscle cells in vivo. For example, a 4:1 ratio mixture of EPCs and human saphenous vein smooth muscle cells (HSVSMC) in extracellular matrix material Matrigel was injected subcutaneously into mice and after a week in vivo, the implant contained numerous small blood vessels which tested positive for specific endothelial cell markers such as CD31 and a-smooth muscle actin (α-SMA).

In one embodiment, human dermal microvascular endothelial cells (HDMEC) or human umbilical vein endothelial cells (HUVEC) can be used with MPCs for neovascularization in vivo.

In another embodiment, human saphenous vein smooth muscle cells (HSVSMC), human brain vascular smooth muscle cells (HBVSMC) Cat. #1100; human esophageal smooth muscle Cells (HESMC) Cat. #2710; human intestinal smooth muscle cells (HISMC) Cat. #2910; human colonic smooth muscle cells (HCSMC) Cat. #2940; human pulmonary artery smooth muscle cells (HPASMC) Cat. #3110; human bronchial smooth muscle cells (HBSMC) Cat. #3400; human tracheal smooth muscle cells (HTSMC) Cat. #3410; human bladder smooth muscle cells (HBdSMC) Cat. #4310; human aortic smooth muscle cells (HASMC) Cat. #6110; human umbilical vein smooth muscle cells (HUVSMC) Cat. #8020; human umbilical artery smooth muscle cells (HUASMC) Cat. #8030 can be used. These cells are commercially available at ScienCell™ Research Laboratories.

### Therapeutic Uses

Encompassed in the invention disclosed herein is the promotion of neovascularization in tissue engineering constructs, tissue repair, regenerative medicine, and wound healing. Tissue engineering is the use of a combination of cells, engineering and material methods, and suitable biochemical and physiochemical factors to improve or replace biological functions. Tissue engineering aims at developing functional cell, tissue, and organ substitutes to repair, replace or enhance biological function that has been lost due to congenital abnormalities, injury, disease, or aging, or repair fascia in hernias. The tissue that is engineered is used to repair or replace portions of or whole tissues (i.e., bone, cartilage, blood vessels, heart valves, bladder, diaphragm, etc.). Often, the tissues involved require certain mechanical and structural properties for proper function. Tissue engineering also encompass the efforts to perform specific biochemical functions using cells within an artificially-created support system (e.g. an artificial pancreas, or a bioartificial liver). The term regenerative medicine is often used synonymously with tissue engineering, although those involved in regenerative medicine place more emphasis on the use of stem cells to produce tissues and on promoting repair in situ. Tissue regeneration aims to restore and repair tissue function via the interplay of living cells, an extracellular matrix and cell communicators.

In vivo therapeutic neovascularization using the invention disclosed herein is contemplated for tissue repair and healing of chronic wound in humans. The human body has a great capacity to heal itself when damaged. However, sometimes, the body's innate healing function becomes impaired or reduced due to metabolic diseases such as diabetes, poor blood circulation, blocked or damaged blood vessels. The invention disclosed herein artificially increases blood vessels in the damaged area, by de novo formation of blood vessels and also stimulates new blood vessels formation from existing ones. The new blood vessels bring oxygen, nutrients and growth factors to stimulate the body's own natural healing process by activating the body's inherent ability to repair and regenerate. In vivo therapeutic neovascularization helps speed up healing and helps injuries that will not heal or repair on their own. In vivo therapeutic neovascularization can be used to heal broken bones, severe burns, chronic wounds, heart damage, nerve damage, damaged tissue of the heart, muscles, skin, adipose tissue, brain, liver, lungs, intestines, limbs, and kidneys to name a few.

In one embodiment, the methods described herein can help cardiac tissue to repair itself weeks after a heart attack. Embryonic stem cells have been shown to regenerate damaged heart muscle, when transplanted within a 3-dimensional scaffold into the infarcted heart. The embryonic stem cells were more successful in restoring heart muscle when transplanted within a 3-dimensional matrix into damaged hearts in an animal model of severe infarction. Instead of embryonic stem cells, a composition comprising EPCs/MPCs (40%:60%) can be placed within a suitable biocompatible scaffold or matrix, and implanted to the infracted heart tissue. In another embodiment, embryonic stem cells or other types of tissue-derived (parenchymal) cells can be used with the composition comprising EPCs/MPCs (e.g. 40%:60%) to seed a suitable biocompatible scaffold or matrix prior to implantation to the tissue repair location. Methods of constructing cardiac related structures are described in U.S. Pat. Nos. 5,880,090, 5,899,937, 6,695,879, 6,666,886, 7,214,371, and US Pat. Publication No. 20040044403 and they are hereby incorporated by reference.

In one embodiment, the composition comprising EPCs/MPCs, can include growth, differentiation, and/or angiogenesis factors that are known in the art to stimulated cell proliferation, differentiation, and angiogenesis the cells at the site where the composition is delivered.

In one embodiment, the composition comprising EPCs/MPCs is directly implanted to the site needing repair, for example, the part of the heart that has suffered a myocardial infarction (Dinender K. Singla, et. al., Am J Physiol Heart Circ Physiol 293: H1308-H1314, 2007). The composition comprising EPCs/MPCs can be injected into the tissue repair site together with growth, differentiation, and angiogenesis factors that are known in the art to stimulated cell growth, differentiation, and angiogenesis in the appropriate cell type of the recipient tissue. Suitable growth factors include but are not limited to transforming growth factor-beta (TGFβ), vascular endothelial growth factor (VEGF), platelet derived growth factor (PDGF), angiopoietins, epidermal growth factor (EGF), bone morphogenic protein (BMP) and basic fibroblast growth factor (bFGF). Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, P A: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag.

In another embodiment, the composition comprising EPCs/MPCs disclosed herein can be implanted in a tissue in need of vascularization by direct injection of the composition. Direct injection is useful for the repair of ischemic tissue, for example, cardiac muscles, blood vessels, kidney, liver, bones, brain the pancreas and the connective and support tissues such as ligaments, muscles, tendons and those tissues, such as the collagen-containing tissues which encapsulate organs, to name a few. Ischemia in a tissue can be determined by methods known to one skilled in the art, such as SPECT and diffusion/perfusion MRI, ankle-brachial index (ABI), Doppler ultrasound, segmental pressures and waveforms, duplex ultrasound, and transcutaneous oxygen pressure. Methods of direct implantation of stem cells for tissue repair are described in Shake J G et, al. 2002 (Ann Thorac Surg. 73:1919-25), Yoshinori Miyaharal, et. al., 2006 (Nature Medicine 12, 459-465), Atta Behfar, et. al., 2005 (Ann. N.Y. Acad. Sci. 1049: 189-198), Luciano C. Amado, et. al., 2005, (PNAS, 102: 11474-9), Khalil P N, et. al., 2007, (Gastroenterology. 132:944-54), Lee R H, et. al., 2006 (Proc Natl Acad Sci U S A.;103:17438-43), and Chamberlain J., et. al., 2007, (Hepatology. 2007Aug. 17, in press), S. P. Bruder, et. al., 1998, (J. Bone and Joint Surgery 80:985-96), Pignataro G., et. al., J. Cereb Blood Flow Metab. 2007 May;27(5):919-27 and are hereby incorporated by reference.

In yet another embodiment, the composition comprising EPCs/MPCs disclosed herein can be ‘seeded’ into an artificial structure capable of supporting three-dimensional tissue formation. These structures, typically called scaffolds, are often critical, both ex vivo as well as in vivo, to recapitulating the in vivo milieu and allowing cells to influence their own microenvironments. Scaffold-guided tissue engineering involves seeding highly porous biodegradable scaffolds with cells and/or growth factors, followed by culturing the tissue engineering constructs in vitro for a time period. Subsequently the scaffolds are implanted into a host to induce and direct the growth of new tissue. The goal is for the cells to attach to the scaffold, then replicate, differentiate, and organize into normal healthy tissue as the scaffold degrades. This method has been used to create various tissue analogs including skin, cartilage, bone, liver, nerve, vessels, to name a few examples. The addition of the EPCs/MPCs mixture promotes the neovascularization of the tissue engineering constructs after implantation in the host.

In one embodiment, a biocompatible scaffold is used in tissue engineering. A scaffold fabricated from biocompatible materials enveloped in a biocompatible material provides an improved substrate for cell attachment. In one embodiment, the biocompatible material used to envelope the scaffold is bioabsorbable. Suitable scaffolds include meshes, other filamentous structures, non-woven, sponges, woven or non-woven materials, knit or non-knit materials, felts, salt eluted porous materials, molded porous materials, 3D-printing generated scaffolds, foams, perforated sheets, grids, parallel fibers with other fibers crossing at various degrees, and combinations thereof. The core scaffold can be in a variety of shapes including sheets, cylinders, tubes, spheres or beads. The core scaffold can be fabricated from absorbable or non-absorbable materials. Suitable absorbable materials include glycolide, lactide, trimethylene carbonate, dioxanone, caprolactone, alklene oxides, ortho esters, polymers and copolymers thereof, collagen, hyaluronic acids, alginates, and combinations thereof. Suitable non-absorbable materials include, polypropylene, polyethylene, polyamide, polyalkylene therephalate (such as polyethylene therephalate polybutylene therephalate), polyvinylidene fluoride, polytetraflouroethylene and blends and copolymers thereof. Suitable biocompatible materials that can be used to envelope the scaffold include absorbable or non-absorbable materials or a combination thereof. Suitable absorbable materials include those stated hereinabove. Suitable non-absorbable materials include those non-absorbable materials stated hereinabove. In some embodiments, the scaffold is embedded or encased in a bioabsorbable material.

Scaffolds can also be constructed from natural materials: in particular different derivatives of the extracellular matrix have been studied to evaluate their ability to support cell growth. Protein based materials, such as collagen or fibrin, and polysaccharidic materials, like chitosan or glycosaminoglycans (GAGs), have all proved suitable in terms of cell compatibility, but some issues with potential immunogenicity still remains. Among GAGs hyaluronic acid, possibly in combination with cross linking agents (e.g. glutaraldehyde, water soluble carbodiimide, etc.), is one of the possible choices as scaffold material. Functionalized groups of scaffolds may be useful in the delivery of small molecules (drugs) to specific tissues.

A variety of scaffolds and uses thereof are described in U.S. Pat. Nos. 6,103,255, 6,224,893, 6,228,117, 6,328,990, 6,376,742, 6,432,435, 6,514,515, 6,525,145, 6,541,023, 6,562,374, 6,656,489, 6,689,166, 6,696,575, 6,737,072, 6,902,932 and WO/2005/110050, they are hereby incorporated by reference.

The procedures for tissue engineering the various tissue types can be found in the methods described in the examples herein, in Koji Kojima, et. al., J. Thorac. Cardiovasc. Surg. 2002, 123:1177-1184, Duxbury M S, et. al., Transplantation, 2004 77:1162-6, U.S. Pat. Nos. 5,700,289, 5,716,404, 6,123,727, 6,171,344, 6,503,273, 6,620,203, 6,666,886, 6,692,761, 6,656,489, 6,840,962, 6,737,053, 7,049,057, 7,049,139, 7,052,514, 7,052,518, 7,112,218, 7,179,287, 7,198,641 and they are hereby incorporated by reference.

Examples of tissue that can be engineered, reconstructed and/or repaired include but are not limited to craniofacial structures such as bone, adipose tissue and facial muscles, cardiac muscle, cardiac valve, skin, bones, skeletal muscles, diaphragmatic muscles and tendons, breast tissue, blood vessels, cartilage, tendons, ligaments, bladder, urether, uterus, ureter, virgina, cervix, trachea, hair, cornea, esophagus and small intestines. Fetal reconstructions of the tracheal and the diaphragm using tissue engineered autologous cartilage grafts and tendons respectively are fully described by Kunisaki et. al., 2006, J. Pediatr. Surg. 41:675-82 and by Fuch et. al., 2004, J. Pediatr. Surg. 39: 834-8 and these are hereby incorporated by reference.

Craniofacial structures reconstruction is the regeneration or de novo formation of dental, oral, and craniofacial structures lost to congenital anomalies, trauma, and diseases. Virtually all craniofacial structures are derivatives of mesenchymal cells. Biological therapies utilize mesenchymal stem cells, delivered or internally recruited, to generate craniofacial structures in temporary scaffolding biomaterials. Several craniofacial structures—such as the mandibular condyle, calvarial bone, cranial suture, and subcutaneous adipose tissue—have been engineered from mesenchymal stem cells, (J. J. Mao, et. al., J Dent Res 85(11):966-979, 2006) and is hereby incorporated by reference.

In one embodiment, the invention disclosed herein can be used to promote wound healing in a human in need thereof comprising delivery of a composition comprising EPCs/MPCs according to the methods described herein.

The composition comprising EPCs/MPCs can be applied directly to wounds to stimulate wound healing. Delivery can be direct injection to the wound, or to the adjacent tissue of the wound. For example, pressure ulcers, leg ulcers, abrasions, lacerations, incisions, donor sites and second degree burns on infected wounds, surgical incisions and traumatic wounds. The composition of EPCs/MPCs can be mixed with growth factors for promoting growth at the site of the wound, and the composition can be applied to the wound. The mixture can also be incorporated into a variety of wound dressing products such as wound dressing gauzes. The application of EPCs/MPCs with or without growth factors help promote healing in areas that may have a reduced capability of self-repair and renewal due to variety of medical conditions such as congestive heart failure, poor circulation, obesity, lymphatic obstructions and diabetes.

In one embodiment, envisioned in the invention is a composition for promoting neovascularization comprising: an enriched population of isolated EPCs; an enriched population of isolated MPCs; and a pharmaceutically acceptable carrier. In one embodiment, the composition comprising a composition of EPCs/MPCs is present in an amount sufficient to promote in vivo neovascularization at the site of implantation, for example, an open wound.

In one embodiment, the EPCs comprise at least 10% but not more than 90% of the total cells in the composition. In another embodiment, the MPCs comprise at least 10% but not more than 90% of the total cells in the composition. In yet another embodiment, the EPCs comprise 40% and the MPCs comprise 60% of the total cells of the composition.

A pharmaceutically acceptable carrier is one that does not cause an adverse physical reaction upon administration and one in which maintains the viability of the EPCs/MPCs for delivery into the patient or use in tissue engineering. In one embodiment, the pharmaceutically acceptable carriers are inherently nontoxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol.

In one embodiment, other ingredients can be added to the pharmaceutical composition, including antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

In one embodiment, the composition of a mixture of EPCs/MPCs should be sterile, is at a physiological pH of between 6-8, and is isotonic to human bodily fluid.

In one embodiment, the composition can include one or more bioactive agents to induce healing or regeneration of damaged cardiac tissue, such as recruiting blood vessel forming cells from the surrounding tissues to provide connection points for the nascent vessels. Suitable bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof.

A great number of growth factors and differentiation factors that are known in the art to stimulated cell growth and differentiation of the progenitor cells. Suitable growth factors and cytokines include any cytokines or growth factors capable of stimulating, maintaining, and/or mobilizing progenitor cells. They include but are not limited to stem cell factor (SCF), granulocyte-colony stimulating factor (G-CSF), granulocyte-macrophage stimulating factor (GM-CSF), stromal cell-derived factor-1, steel factor, vascular endothelial growth factor (VEGF), TGFβ, platelet derived growth factor (PDGF), angiopoeitins (Ang), epidermal growth factor (EGF), bone morphogenic protein (BMP), fibroblast growth factor (FGF), hepatocye growth factor, insulin-like growth factor (IGF-1), interleukin (IL)-3, IL-1α, IL-1β, IL-6, IL-7, IL-8, IL-11, and IL-13, colony-stimulating factors, thrombopoietin, erythropoietin, fit3-ligand, and tumor necrosis factor α. Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, P A: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag.

In one embodiment, the composition of the invention is a suspension of progenitor cells in a suitable physiologic carrier solution such as saline. The suspension can contain additional bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof.

In another embodiment, the composition of the invention is a suspension of progenitor cells in gel-like components of the extracellular matrix. Components of the extracellular matrix comprise of fibrous proteins and polysaccharides, for example, glycosaminoglycans (GAGs), proteoglycans, heparan sulfate proteoglycans, chondroitin sulfate proteoglycans, keratan sulfate proteoglycans, hyaluronic acid, elastin, collagen, fibronectin, and laminin. In another embodiment, the composition of the invention is a suspension of progenitor cells in poly-lysine. The gel-like composition holds the progenitor cells in 3-dimensional space at the site of application on the tissue engineered construct or at the site of tissue repair. This prevents random diffusion of the cells and washing away of cells before they have a chance to adhere to the tissue engineered construct or tissue needing repair. The suspension can contain additional bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof. Examples of growth factors that can be used in a matrix comprising laminin, collagen IV and entactin, are EGF, bFGF, NGF, PDGF, IGF-1and TGF-β. An example of such a gel-like composition is a matrix comprising laminin (56%), collagen IV (31%) and entactin (8%), EGF (0.5-1.3 ng/ml), bFGF (<0.1-0.2 pg/ml), NGF (<0.2 ng/ml), PDGF (5-48 pg/ml), IGF-1 (11-24 ng/ml), and TGF-β (1. 7.7 ng/ml).

In another embodiment, the composition of the invention is a wound dressing material impregnated with isolated EPCs and MPCs. The EPCs and MPCs are embedded in a wound dressing material such as a gauze and the seeded wound dressing material is applied on to a chronic wound. Examples of other wound dressing materials include, for example, alginates, composites, exudate absorbers, foams, hydrocolloids, hydrogels, skin sealants, transparent films, the 3M Hydrogels, water soluble wound dressing materials described in U.S. Pat. No. 4,233,969, swellable wound dressing materials described in U.S. Pat. No. 6,022,556, and active wound dressing materials described in the International Patent Application Publication WO 2007068885, and these are hereby incorporated by reference. In another embodiment, the seeded wound dressing material can contain additional bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof.

In one embodiment, the quantity of progenitor cells delivered in the composition disclosed herein to an tissue engineered construct or a tissue in need will vary based on the individual patient, the size of the construct or tissue or wound, the thickness of the construct, the number of sites for delivery within the tissue, wound, or adjacent tissue, the indication being treated and other criteria evident to one of ordinary skill in the art. Additionally, the frequency of deliver also can vary. A therapeutically effective amount of progenitor cells in the composition is one sufficient to bring about neovascularization to the tissue engineered construct and/or a target organ or tissue. In one embodiment, 1×10⁴ to 1×10⁹ total progenitor cells are delivered in the composition. For tissue engineered constructs, at least 1×10⁶ total progenitor cells per 1 ml volume is recommended. For therapeutic neovascularization in tissue repair, the precise determination of the amount of cells is based on factors individual to each patient, including their weight, age, size of the treatment area, and the amount of time since ischemic injury. The person of ordinary skill in the art can also readily determine the dosage of cells, amount of composition, type of pharmaceutically acceptable carrier and other bioactive agents to be delivered based on the present disclosure and the general knowledge known in the art.

The method of delivering the composition comprising EPCs and MPCs cells also vary based on the individual patient, the indication being treated and other criteria evident to one of ordinary skill in the art. The route(s) of delivery useful in a particular application are apparent to one of ordinary skill in the art. Routes of administration include, but are not limited to, topical, transdermal, and direct injection to the specific tissue site or organ. Topical and transdermal delivery is accomplished via a wound dressing impregnated with a composition of EPCs and MPCs, or the gel-like matrix suspension of progenitor cells, allowing the progenitor cells to migrate and enter the wound and also enter the blood stream. Direct injection delivery methods, including intramuscular, intracoronary and subcutaneous injections, can be accomplished using a needle and syringe, using a high pressure, needle free technique, like POWDERJECT™, constant infusion pump, a catheter delivery system, or the injection apparati disclosed in the International Patent Publication number WO 2007112136.

In one embodiment, the total volume of the composition comprising EPCs and MPCs injected into tissue for therapeutic neovascularization is limited to 1 ml per injection site. The volumes injected can vary from the range of 50 μl to 1 ml. In one embodiment, several injection sites are selected within the tissue in need of neovascularization. This ensure even neovascularization of the target tissue or chronic wound and promote faster neovascularization. Volumes ranging from 50 μl to 1 ml can be injected at each site. Generally, the closer the sites of injection are together, the smaller the amount of the composition disclosed herein is delivered to each site. A physician skilled in the art can decide on the number of inject sites and the frequency of delivery depending on the tissue or chronic wound needing ascularization. Example of direct localized delivery of therapeutics to the cardiac muscles is described in the International Patent Publication number WO 2007112136 and is hereby incorporated by reference.

In one embodiment, the enriched populations of isolated EPCs and isolated MPCs are delivered simultaneously to each site of delivery by methods disclosed herein and known in the art. The EPCs and MPCs can be mixed in the recommended ratio as described herein and the mixture of progenitor cells is then delivered using a single needle and syringe at the injection site. Alternately, a multi-chambered needle-syringe, as described in the International Patent Publication number WO 2007112136, can be use for delivering the EPCs and MPCs simultaneously. Separate chamber holds a different progenitor cell type. When the syringe plungers are depressed, the different progenitor cell type enters a common chamber, and is mixed prior to delivery into the injection site. The depression of the syringe plunger can be automated to depress at different rates in order to achieve the recommended ratios of EPCs to MPCs as disclosed herein. In another embodiment, the enriched populations of isolated EPCs and isolated MPCs are delivered sequentially. Separate single-chambered needle-syringes can be used for delivery to a single injection site.

Envisioned in the invention is a kit comprising: an isolated enriched population of endothelial progenitor cells; and an isolated enriched population of mesenchymal progenitor cells. In one embodiment, the kit further comprises an extracellular matrix or a biocompatible scaffold. In one embodiment, the kit further comprises an assortment of bioactive agents as disclosed herein to aid in cell growth, migration, and differentiation. In another embodiment, the kit also provides instructions for using the EPCs, MPCs, extracellular matrix, biocompatible scaffold, and bioactive agents to achieve neovascularization in ischemic tissues and organs, and tissue engineered constructs.

This invention is further illustrated by the following example which should not be construed as limiting. The contents of all references cited throughout this application, as well as the figures and tables are incorporated herein by reference.

### Example 1

In Vivo Vasculogenic Potential of Human Blood-Derived Endothelial Progenitor Cells (EPC).

Materials and Methods.

Isolation and culture of blood-derived EPCs—Human umbilical cord blood was obtained from the Brigham and Women's Hospital in accordance with an Institutional Review Board-approved protocol. Adult peripheral blood was collected from volunteer donors in accordance with a protocol approved by Children's Hospital Boston Committee on Clinical Investigation. Both cord blood-derived EPCs (cbEPCs) and adult peripheral blood-derived EPCs were obtained from the mononuclear cell (MNC) fractions similarly to other authors (Ingram D A, et. al., Blood. 2004,104:2752-60; Lin Y, et. al., J Clin Invest. 2000,105:71-77; Yoder M C, et. al., Blood. 2006, 109:1801-9). MNCs were seeded on 1% gelatin-coated tissue culture plates using Endothelial Basal Medium (EBM-2) supplemented with SingleQuots (except for hydrocortisone) (Cambrex BioScience, Walkersville, Md.), 20% FBS (Hyclone, Logan, Utah), 1× glutamine-penicillin-streptomycin (GPS; Invitrogen, Carlsbad, Calif.) and 15% autologous plasma (Wu X, et. al., Am J Physiol Heart Circ Physiol. 2004, 287:H480-487). Unbound cells were removed at 48 hours for cord blood and at 4 days for adult blood. In both cases, the bound cell fraction was then maintained in culture using EBM-2 supplemented with 20% FBS, SingleQuots (except for hydrocortisone) and 1× GPS (this medium is referred to as EBM-2/20%). Colonies of endothelial-like cells were allowed to grow until confluence, trypsinized and purified using CD31-coated magnetic beads (Dynal Biotech, Brown Deer, Wis.). CD31-selected EPCs were serially passaged and cultured on fibronectin-coated (FN; 1 ug/cm²; Chemicon International, Temecula, Calif.) plates at 5×10³ cell/cm² in EBM-2/20%. HDMECs from newborn foreskin cultured in the same condition as cbEPCs were used as positive controls (Kraling B M, et. al., In Vitro Cell Dev Biol Anim. 1998;34:308-315). Human saphenous vein smooth muscle cells (HSVSMCs) grown in DMEM (Invitrogen), 10% FBS, 1× GPS and 1× Non essential amino acids (Sigmna-Aldrich, St. Louis, Mo.) were used as negative controls for endothelial phenotype.

Phenotypic characterization of cbEPCs—Cytometric analyses were carried out by labeling with phycoerythrin (PE)-conjugated mouse anti-human CD31 (Ancell, Bayport, Minn.), PE-conjugated mouse anti-human CD90 (Chemicon International), fluorescein isothiocyanate (FITC)-conjugated mouse anti-human CD45 (BD PharMingen, San Jose, Calif.), FITC-mouse IgG1 (BD PharMingen), PE-mouse IgG1 (BD PharMingen) antibodies (1:100), PE-conjugated mouse anti-human CD105 (1:50; Serotec, Raleigh, N.C.), PE-conjugated mouse anti-human CD44 (1:100; BD PharMingen), FITC-conjugated mouse anti-human CD29 (1:100; Immunotech/Beckman Coulter, Fullerton, Calif.), PE-conjugated mouse anti-human CD34 (1:50; Miltenyi Biotec, Auburn, Calif.), PE-conjugated mouse anti-human VEGF-R2 (1:50; R&D Systems, Minneapolis, Minn.), PE-conjugated mouse anti-human Neuropilin-1 (1:100; Miltenyi Biotec), FITC-conjugated mouse anti-human CD146 (1:100; Chemicon International), and FITC-conjugated mouse anti-human CD14 (1:100; BD PharMingen). Human dermal microvascular ECs (HDMECS) from newborn foreskin, SMCs from human saphenous vein, and adult peripheral blood monocytes (pbMonocytes) served as controls. Antibody labeling was carried out for 20 minutes on ice followed by 3 washes with PBS/1% BSA/0.2 mM EDTA and resuspension in 1% paraformaldehyde in PBS. Flow cytometric analyses were performed using a Becton Dickinson FACScan flow cytometer and FlowJo software (Tree Star Inc., Ashland, Oreg.).

Indirect immunofluorescence—Immunofluorescence was carried out using goat anti-human CD31 (1:200; Santa Cruz Biotechnology), mouse anti-human vWF (1:200; DakoCytomation), goat anti-human VE-cadherin (1:200; Santa Cruz Biotechnology), mouse anti-human α-smooth muscle actin (1:2000; α-SMA; Sigma-Aldrich), mouse anti-human Calponin (1:100; DakoCytomation), mouse anti-human smooth muscle myosin heavy chain (1:100; Sigma-Aldrich), and mouse anti-human NG2 (1:100; Sigma-Aldrich) antibodies, followed by FITC-conjugated secondary antibodies (1:200; Vector Laboratories) and Vectashield mounting medium with DAPI (Vector Laboratories).

In Vitro Maturation of cbEPCs

Expansion potential of cbEPCs-cbEPCs and adult EPCs, were isolated as described above and expanded for 112 and 60 days, respectively. All passages were performed by plating the cells onto 1 μg/cm² FN-coated tissue culture plates at 5×10³ cell/cm² using EBM-2/20%. Medium was refreshed every 2-3 days and cells were harvested by trypsinization and re-plated in the same culture conditions for the next passage. Cumulative values of total cell number were calculated by counting the cells at the end of each passage using a haemocytometer.

Growth kinetics assay—Growth curves of cbEPCs were evaluated at different passages. Cells were plated in triplicates onto 1 μg/cm² FN-coated 24-well tissue culture plates at 5×10³ cell/cm² in 0.5 ml of EBM-2/20%. Medium was refreshed every two days and cell numbers evaluated at 24 hour intervals for 7 days by counting the cells after trypsinization using a haemocytometer. Doubling time profiles were calculated from the mean values obtained from each growth curve at different passages (Melero-Martin J M, et. al., Biotechnol Bioeng. 2006, 93:519-533).

Cell size measurements—Morphological differences of cbEPCs were evaluated at different passages. Confluent cell monolayers were immunostained with VE-cadherin antibody for cell surface and DAPI for nuclear visualization as described above. The areas occupied by cell bodies and cell nuclei were measured by analysis (ImageJ software, NIH) of the images obtained from randomly selected fields from three separate cultures after immunostaining. All values were normalized to the value of total cell area.

Proliferation assay—Cells were seeded in triplicates onto 1 μg/cm² FN-coated 24-well plates at 5×10³ cell/cm² using EMB-2 supplemented with 5% FBS and 1× GPS (control medium); plating efficiency was determined at 24 hours, then cells were treated for 48 hours using control medium in the presence or absent of either 10 ng/ml of VEGF-A (R&D Systems) or 1 ng/ml bFGF (Roche Applied Science, Indianapolis, Ind.). Cells were trypsinized and counted using a haemocytometer. Values were normalized to the cell numbers determined at 24 hours.

In Vivo Vasculogenesis Experiments

Matrigel implantations—Unless otherwise indicated, 1.5×10⁶ EPCs were mixed with 0.375×10⁶ HSVSMCs (4:1 ratio) and resuspended in 200 μl of Phenol Red-free Matrigel (BD Bioscience, San Jose, Calif.) on ice. The mixture was implanted on the back of a six-week-old male athymic nu/nu mouse (Charles River Laboratories, Boston, Mass.) by subcutaneous injection using a 25-gauge needle. One implant was injected per mouse. Each experimental condition was performed with 4 mice.

Histology and immunohistochemistry—Matrigel implants were removed at one week after xenografting, fixed in 10% buffered formalin overnight, embedded in paraffin, and sectioned. Hematoxylin and eosin (H&E) stained 7 μm-thick sections were examined for the presence of lumenal structures containing red blood cells. For immunohistochemistry, 7-μm-thick sections were deparaffinized, blocked for 30 minutes in 5% horse serum, and incubated with human-specific CD31 monoclonal antibody (1:50, DakoCytomation), anti-human α-SMA (1:750, Sigma-Aldrich), or mouse IgG (DakoCytomation) for 1 hour at room temperature. Horseradish peroxidase-conjugated secondary antibody and 3,3′-diaminobenzidine (DAB) were used for detection. The sections were counterstained with hematoxilin and mounted using Permount (Fisher Scientific).

Microvessel density analysis—Microvessels were detected by the evaluation of H&E stained sections taken from the middle part of the implants. The full area of each individual section was evaluated. Microvessels were identified and counted as lumenal structures containing red blood cells. The area of each section was estimated by image analysis. Microvessels density was calculated by dividing the total number of red blood cell-filled microvessels by the area of each section (expressed as vessels/mm²). Values reported for each experimental condition correspond to the average values obtained from four individual animals.

Statistical analysis—The data were expressed as means ±SD. Where appropriate, data were analyzed by analysis of variance (ANOVA) followed by two-tailed Student's unpaired t-tests. P value<0.05 was considered to indicate a statistically significant difference.

Results.

Phenotypic characterization of cbEPCs—The EPCs isolated from the MNC fraction of human umbilical cord blood samples (n=19) were similarly to other authors (Ingram D A, 2004, and Lin Y, 2000). Cord blood-derived endothelial colonies (identified by typical cobblestone morphology) emerged in culture after one week. The size, frequency, and time of appearance of these colonies varied as already reported by Ingram D A, 2004, (data not shown). Endothelial colonies were left to grow in the original culture plates until confluence and purified thereafter (at passage 1) by selection of CD31-positive cells. This procedure resulted in superior cell yields compared to our previous isolation protocol based on double selection of CD34+/CD133+ cells from the MNC fraction (Wu X, 2004). However, since CD31 is not a specific marker of EPCs and due to the heterogeneity of blood preparations, both phenotypical and functional characterization were performed. This was especially important considering that earlier studies have shown that some EPC colonies isolated from MNCs contain cells that express the hematopoietic-specific cell-surface antigen CD45 (Rafii S, Lyden D, Nat Med. 2003, 9:702-712; Rehman J, et. al., Circulation. 2003, 107:1164-1169; Gulati R, et. al., Circ Res. 2003;93:1023-1025), raising questions about the cellular origin of circulating EPCs.

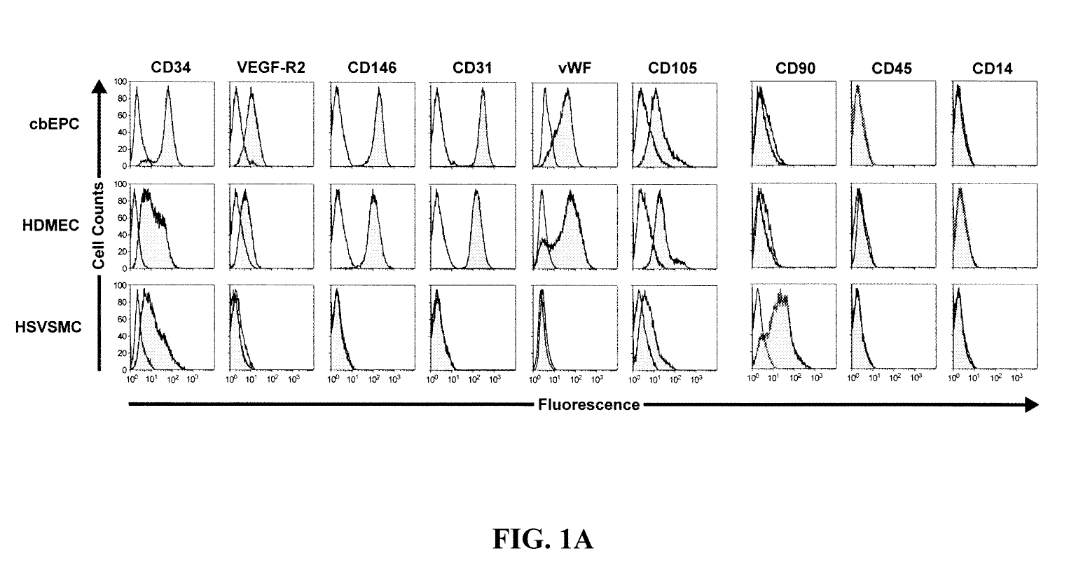

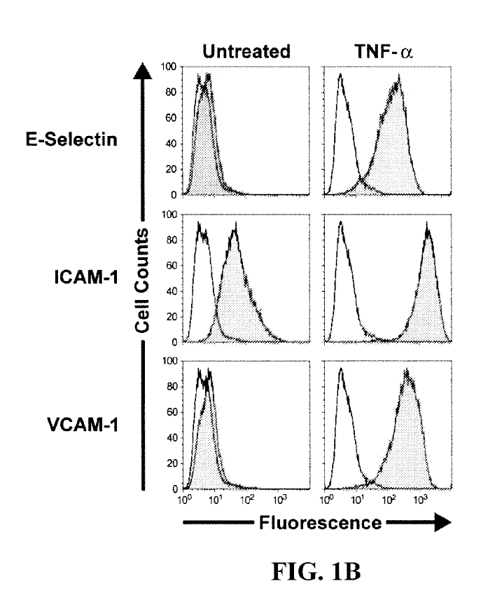

The endothelial phenotype of the isolated cbEPCs was confirmed by different methods. Flow cytometric analysis of cbEPCs showed remarkably uniform expression of EC markers CD34, VEGF-R2, CD146, CD31, vWF and CD105 (FIG. 1A). In addition, cells were negative for mesenchymal marker CD90 and hematopoietic markers CD45 and CD14, confirming that the cells were not contaminated with either mesenchymal or hematopoietic cells. Additionally, RT-PCR analyses showed the expression of EC markers CD34, VEGF-R2, CD31, VE-cadherin, vWF and eNOS at the mRNA level (data not shown). Indirect immunofluorescent staining was performed to further examine the expression of EC markers. The results showed that cbEPCs expressed CD31, VE-cadherin and vWF (data not shown). Importantly, the localization of CD31 and VE-cadherin at the cell-cell borders and vWF in a punctuate pattern in the cytoplasm showed clear indications of EC properties.

In addition, the cbEPCs were tested for the ability to up-regulate leukocyte adhesion molecules in response to the inflammatory cytokine TNF-α. The low-to-undetectable levels of E-selectin, ICAM-1 and VCAM-1 in the untreated cbEPC cultures were up-regulated upon 5 hour incubation with TNF-α (FIG. 1B). This response to an inflammatory cytokine is characteristic of ECs and suggests that the use of cbEPC in the formation of microvascular vessels could also provide physiologic proinflammatory properties.

In summary, this combination of analyses provides a definitive demonstration that the cells isolated from umbilical cord blood were ECs and discards the possibility of hematopoietic/monocytic cells in the culture (Yoder M C, 2007). Based on the isolation methodology and the phenotypical characteristics, the isolated EPCs are similar to those referred to by other authors as late-EPCs or endothelial outgrowth cells (Lin Y, 2000; Gulati R, 2003). The characterization depicted in FIG. 1 corresponded to cbEPCs at passage 6. A detailed characterization was performed at passages 4, 9, 12 and 15 with similar results (data not shown), indicating a stable endothelial phenotype through long term culture. Furthermore, additional characterization of the cbEPCs at passage 6 showed that cbEPCs express two other VEGF-receptors, neuropilin-1 and Flt-1, and that the cbEPCs do not express the smooth muscle/mesenchymal cell markers PDGF-Rβ, α-SMA, or calponin (data not shown).

In vivo vasculogenic potential of cbEPCs—To determine whether cbEPCs were capable of forming functional capillary networks in vivo, cbEPCs, implanted in Matrigel, were placed subcutaneously into nude mice for one week. For this experiment, 1.5×10⁶ of cbEPCs (passage 6) were combined with 0.375×10⁶ HSVSMCs in 200 μl of Matrigel, resulting in a ratio of cbEPCs to HSVSMCs of 4 to 1, and injected subcutaneously. This ratio of cbEPCs to HSVSMCs was less than the 1:1 ratio previously used by Wu X, 2004, with the intention to minimize the contribution of smooth muscle cells. After harvesting the Matrigel implants, H&E staining revealed the presence of lumenal structures containing murine erythrocytes throughout the implants (data not shown). Similar results were obtained with cbEPCs isolated from three different cord blood samples, yielding an average of 47.5±8 microvessels/mm² (data not shown). Importantly, implants with either cbEPCs or HSVSMCs alone failed to form any detectable microvessels after one week. Injections of Matrigel alone resulted in the appearance of few host cells infiltrated into the borders of the implants, indicating that Matrigel itself was not responsible for the presence of vascular structures within the implants.

To further characterize the microvascular structures detected, sections of the implant were immunohistochemically stained using a human-specific CD31 antibody. Nearly all of the lumenal structures stained positive for human CD31, confirming that those lumens were formed by the implanted human cbEPCs and not by the host cells (data not shown). This result was important because it demonstrated that the formation of microvascular vessels within the implant is the result of a process of in vivo vasculogenesis carried out by the implanted cells and it is not due to blood vessel invasion and sprouting, i.e., an angiogenic response from nearby host vasculature. The specificity of the anti-human CD31 antibody (Parums D V, J Clin Pathol. 1990, 43:752-757; Levenberg S, Proc Natl Acad Sci U S A. 2003;100:12741-12746) was confirmed by the negative reaction obtained when mouse lung tissue sections were stained in parallel (data not shown). Taken together, the human endothelial identity of the lumenal structures and the presence of murine erythrocytes within those structures, it was evident that vasculogenesis occurred and, in addition, the newly created microvessels formed functional anastomoses with the host circulatory system. Next, the time course of vasculogenesis in the Matrigel was analyzed by harvesting implants at 2, 4 and 7 days after xenografting. At 2 days, a low degree of cellular organization was seen (data not shown). At 4 days, a high degree of organization with clear alignment of cells throughout the implant was observed, suggesting formation of cellular cords. The presence of functional microvascular vessels, defined by the presence of red blood cells within the lumen, was appreciable one week after implantation.

The location of the HSVSMCs was also examined by immunohistochemical staining using anti-α-SMA. Smooth muscle cells were detected both around the lumenal structures and throughout the Matrigel implants (data not shown), suggesting an ongoing process of vessel maturation and stabilization (Folkman J, Cell. 1996, 87:1153-5; Darland D C, J Clin Invest. 1999, 103:157-8; Darland D C, Curr Top Dev Biol. 2001,52:107-149). However, the α-SMA antibody is not human-specific, as shown by the positive staining of control tissue sections obtained from mouse lung (data not shown). Therefore, the observed α-SMA positive cells could corresponded to the implanted HSVSMCs or murine cells recruited from the host, or a combination of these.

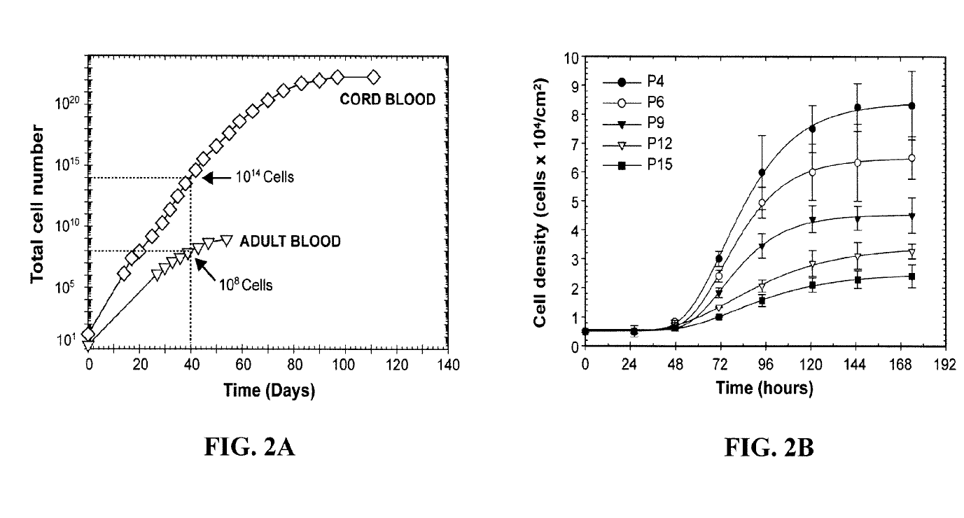

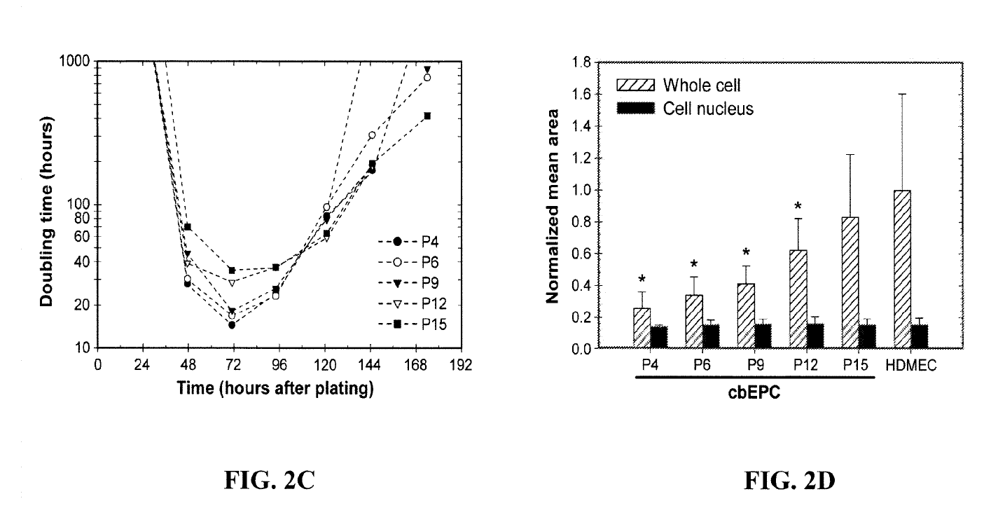

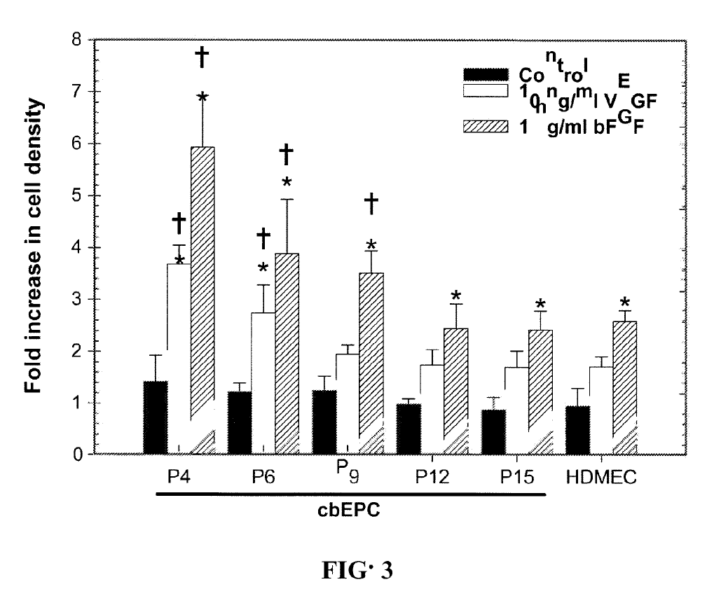

Maturation of cbEPC during in vitro expansion—cbEPCs were serially passaged to determine their expansion potential. Remarkably, 10¹⁴ cells could theoretically be obtained after only 40 days in culture, and thereafter cells were expanded up to 70 population doublings (FIG. 2A), which is consistent with previous studies (Ingram D A, 2004). Significant expansion of adult blood EPCs (10⁸ cells) was also achieved under the same conditions using 50 milliliters of adult peripheral blood (FIG. 2A). In addition to this enormous proliferative capacity, cbEPCs expressed and maintained a definitive endothelial phenotype in vitro as shown in FIG. 1. However, neither the expansion potential nor the phenotypical stability rules out the possibility of cbEPCs undergoing cellular changes during their expansion in vitro. To investigate potential changes, the growth kinetics of cbEPCs at different passages were examined by the generation of growth curves (FIG. 2B), the cells from earlier passages presented superior growth kinetics and reached higher cell densities at confluence. The former was confirmed by the generation of the doubling time profiles (FIG. 2C), where lower passage number corresponded with shorter doubling times. The u-shape of these profiles is the result of mechanisms controlling cell growth in vitro: longer doubling times were found during both the early and late stages of the culture corresponding to the initial lag phase and the inhibition of cell growth by cell-cell contacts, respectively. Taking the minimum values as representative of the dividing capacity, cbEPCs presented minimum doubling times of 14, 17, 18, 29 and 35 hours at passages 4, 6, 9, 12, and 15 respectively. These results illustrated the remarkable dividing capacity of cbEPCs at low passage numbers, and showed that as cbEPCs were expanded in vitro, their growth kinetics progressively slowed.

Serially passaging of cbEPCs also resulted in evident morphological differences. As they were expanded, cells progressively occupied larger areas in culture (FIG. 2D). While the areas occupied by the cell nuclei remained constant at each passage, cbEPCs were found to be significantly (P<0.05) smaller than the control HDMECs, with the exception of passage 15. As cbEPCs were expanded in vitro, the average area occupied by the cells increased towards that of HDMECs. The mean area of cbEPCs ranged from values 75% smaller than HDMECs at passage 4 to 17% smaller at passage 15. These results were consistent with the differences found in cell density at confluence (FIG. 2B).

Next, the proliferative responses of cbEPCs at different passages to stimulation by angiogenic factors VEGF or bFGF were studied (FIG. 3). After the initial 24 hour period, cells were treated with control medium in the presence or absent of either 10 ng/ml of VEGF or 1 ng/ml bFGF and assayed for cell number after 48 hours. Both angiogenic factors produced a proliferative response in all the cases evaluated as compared to basal proliferation in the presence of 5% serum (control). The response was statistically significant (P<0.05) in all the groups treated with bFGF. Interestingly, the proliferative response to bFGF was progressively reduced as passage number increased, and ranged from 5.4-fold at passage 4 to 2-fold at passage 15. When compared to HDMECs, the response toward bFGF was found significantly higher in cbEPCs at passages 4, 6 and 9, but not in the later passages. In the case of VEGF treatment, the response was statistically significant (P<0.05) at passages 4 and 6 as compared to basal proliferation. Again, the proliferative response was progressively reduced as passage number increased, and varied from 3.1-fold in the earliest passage to 1.3-fold in the latest passage group. Collectively, these in vitro experiments demonstrate that despite the consistent and stable expression of endothelial markers, cbEPCs undergo cellular and functional changes as they are expanded in culture. Their morphology, growth kinetics and proliferative responses toward angiogenic growth factors progressively resembled those of HDMECs, indicating a process of in vitro cell maturation over time. It has been showed previously that proliferative responses of HDMECs isolated in our laboratory do not change from passage 3-12 (Kraling B M, 1998).

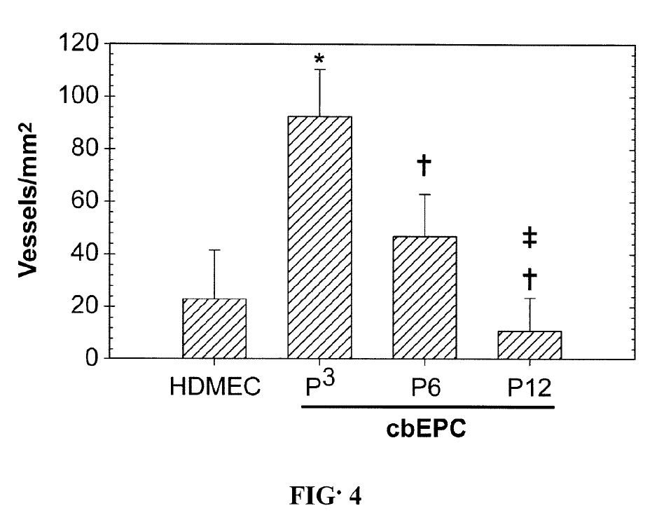

Effect of in vitro expansion of cbEPCs on in vivo vasculogenesis—To answer this question, cbEPCs at different passages (3, 6, and 12) were implanted subcutaneously into nude mice in the presence of HSVSMCs. Examination after one week of the H&E-stained implants (Data not shown) revealed a difference in the level of in vivo neovascularization. Quantification of the red blood cell-containing microvessels (FIG. 4) showed that the differences among the groups were statistically significant (P<0.05) in all the cases, with values ranging from 93±18 vessels/mm² when using cbEPCs at passage 3 to 11±13 vessels/mm² with passage 12. These results show that expansion of the cell population in vitro has indeed a significant impact in the subsequent performance in vivo. Parallel evaluation using mature HDMECs also revealed the presence of 23±19 vessels/mm². This number of microvessels was inferior to those generated by the earliest passages of cbEPCs (passages 3 and 6), with values significantly higher in the case of cbEPCs at passage 3. In contrast, HUVECs combined with HSVSMCs formed 52±9 vessels/mm² (data not shown), indicating a robust vasculogenic potential from this source of ECs.

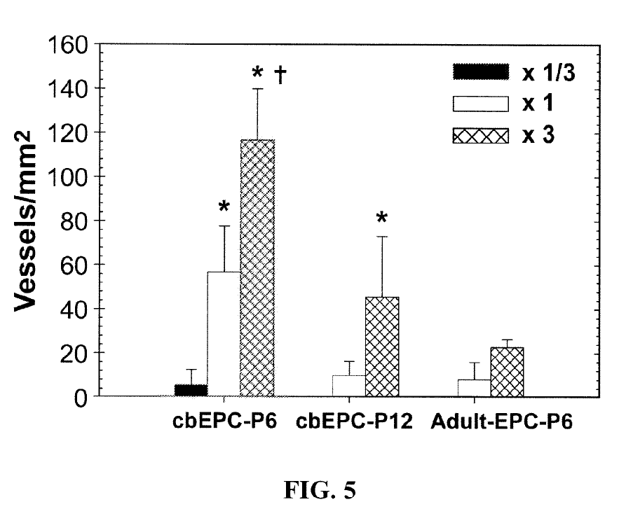

To evaluate whether the lower vasculogenic ability observed in expanded cbEPCs could be compensated by increasing the initial number of EPCs seeded used in the implants, either 0.5×10⁶ (referred to as ×1/3), 1.5×10⁶ (×1) or 4.5×10⁶ (×3) cbEPCs at passages 6 and 12 (FIG. 5) was implanted in the presence of HSVSMCs at a constant 4:1 ratio. One week after xenografting, examination of the H&E-stained implants (data not shown) revealed that an increase in the number of cbEPCs resulted in a higher degree of in vivo neovascularization. Quantification of the microvessel densities (FIG. 5) showed that the differences among the groups of cbEPCs at passage 6 were statistically significant (P<0.05), with values ranging from 6±7 vessels/mm² to 117±23 vessels/mm² when using ×1/3 or ×3 respectively. Consistent with the previous results (FIG. 4), the values of microvessel density in implants of cbEPCs at passage 6 were always higher than those at passage 12 when the same numbers of cbEPCs were used; indeed no microvessels were detected with ×1/3 passage 12 cells. Nevertheless, at passage 12, the partial loss of vasculogenic potential was compensated by increasing the number of seeded cells. As seen in FIG. 5, by simply seeding the implants with 3 times higher density of cbEPCs at passage 12, microvessel density was raised from 10±6 vessels/mm² (×1) to 46±28 vessels/mm² (×3). Furthermore, the microvessel level achieved with ×3 cells passage 12 cells was similar to the level achieved with passage 6 cells at ×1 (P=0.56).

To test whether a similar approach (i.e., increasing the number of EPCs seeded) would result in increased vasculogenesis when using EPCs isolated from blood of adult volunteers, either 1.5×10⁶ (×1) or 4.5×10⁶ (×3) adult EPCs at passages 6 was implanted in the presence of HSVSMCs (4:1 ratio). One week after xenografting, examination of the H&E-stained sections and human CD31-specific immunostaining revealed the presence of human microvessels containing red blood cells in both cases. As occurred with cbEPCs, an increase in the number of adult EPCs resulted in a higher degree of in vivo neovascularization with values ranging from 8±8 lumens/mm² to 23±4 lumens/mm² when using ×1 or ×3 adult EPCs respectively. Quantification of the microvessel densities (FIG. 5) showed that adult EPCs at ×3 was similar to cbEPC-P6 ×1 (P=0.10) and cbEPC-P12 ×3 (P=0.2). In summary, these in vivo experiments clearly show that in addition to the cellular and functional changes observed in vitro, the vasculogenic ability of expanded EPCs progressively diminished but that this effect can be compensated by increasing the number of EPCs initially seeded in Matrigel.

### Example 2

Engineering Vascular Networks In Vivo with Human Postnatal Progenitor Cells Isolated From Blood and Bone Marrow.

Materials and Methods

Isolation and culture of EPCs—EPCs from human umbilical cord blood and adult peripheral blood were isolated and cultured as described above.

Isolation and culture of MPCs—bmMPCs were isolated from the MNC fractions of a 25 mL human bone marrow sample (Cambrex Bio Science, Walkersville, Md.). MNCs were seeded on 1% gelatin-coated tissue culture plates using EGM-2 (except for hydrocortisone, VEGF, bFGF, and heparin), 20% FBS, 1× GPS and 15% autologous plasma. Unbound cells were removed at 48 hours, and the bound cell fraction maintained in culture until 70% confluence using MPC-medium: EGM-2 (except for hydrocortisone, VEGF, bFGF, and heparin), 20% FBS, and 1× GPS. Commercially available bmMPCs (Cambrex) were used as control to those isolated in our laboratory. Similarly, cbMPCs were isolated from the MNC fractions of 25 mL human cord blood samples (n=5). Unbound cells were removed at 48 hours, and the bound cell fraction maintained in culture using MPC-medium. cbMPCs emerged in culture forming mesenchymal-like colonies after one week. These cbMPCs colonies were selected with cloning rings and cultured separately from the rest of the adherent cells. Then after, both bmMPCs and cbMPCs were subcultured routinely on FN-coated plates using MPC-medium. MPCs between passages 4 and 9 were used for all the experiments.

Cell expansion potential—cbEPCs and MPCs were isolated from 25 mL of either cord blood or bone marrow samples and serially expanded in culture using EPC-medium and MPC-medium respectively. All passages were performed by plating the cells onto 1 μg/cm2 FN-coated tissue culture plates at either 5×10³ cell/cm² (cbEPCs) or 1×10⁴ cell/cm² (MPCs). Medium was refreshed every 2-3 days and cells were harvested by trypsinization and re-plated using the same culture conditions for each passage. Cumulative values of total cell number were calculated after 25, 40 and 60 days in culture by counting the cells at the end of each passage using a haemocytometer.

Flow cytometry—Cytometric analyses were carried out as described above.

Western blot—Cells were lysed with 4 mol/L urea, 0.5% SDS, 0.5% NP-40, 100 mmol/L Tris, and 5 mmol/L EDTA, pH 7.4, containing a protease inhibitor cocktail Complete Mini tablet (Roche Diagnostics, Indianapolis, Ind.). Lysates were subjected to 10% SDS-PAGE (10 μg of protein per lane) and transferred to Immobilon-P membrane. Membranes were incubated with respective primary antibodies, goat anti-human CD31 (1:500; Santa Cruz Biotechnology, Santa Cruz, Calif.), goat anti-human VE-cadherin (1:10,000; Santa Cruz Biotechnology), mouse anti-human α-SMA (1:2000; Sigma-Aldrich, St. Louis, Mo.), mouse anti-human calponin (1:500; Sigma-Aldrich), and mouse anti-human β-actin (1:10,000; Sigma-Aldrich) diluted in 1× PBS, 5% dry milk, 0.1% Tween-20, and then with secondary antibodies (1:5000; peroxidase-conjugated anti-goat or anti-mouse; Vector Laboratories, Burlingame, Calif.). Antigen-antibody complexes were visualized using Lumiglo and chemiluminescent sensitive film. SMCs isolated from human saphenous veins and grown in DMEM, 10% FBS, 1× GPS, and 1× non-essential amino acids served as control.

Western blot analysis of PDGF-Rβ—MPCs were plated at a density of 1×10⁴ cell/cm² onto FN-coated plates and cultured using DMEM, 10% FBS, and 1× GPS in the presence or absence of TGF-β1 (2 ng/ml), PDGF-BB (50 ng/ml), and a combination of TGF-β1+PDGF-BB (2 ng/ml+50 ng/ml). Cell lysates were harvested after 6 days and Western blot analysis carried out using goat anti-human PDGF-Rβ (1:250; Santa Cruz Biotechnology), mouse anti-human β-actin (1:10000; Sigma-Aldrich) and peroxidase-conjugated anti-goat or anti-mouse secondary antibodies (1:5000; Vector Laboratories). SMCs served as control. Quantification was performed by image analysis of the bands (ImageJ software; NIH, Bethesda, Md.).

Osteogenesis assay—Confluent MPCs were cultured for 10 days in DMEM low-glucose medium with 10% FBS, 1× GPS, and osteogenic supplements (1 μM dexamethasone, 10 mM β-glycerophosphate, 60 μM ascorbic acid-2-phosphate). Differentiation into osteocytes was assessed by alkaline phosphatase staining (Pittenger, M. F. et al., 1999, Science 284, 143-147).

Chondrogenesis assay—Suspensions of MPCs were transferred into 15 ml polypropylene centrifuge tubes (500,000 cells/tube) and gently centrifuged. The resulting pellets were statically cultured in DMEM high-glucose medium with 1× GPS, and chondrogenic supplements (1× insulin-transferrin-selenium, 1 μM dexamethasone, 100 μM ascorbic acid-2-phosphate, and 10 ng/mL TGF-β1). After 14 days, pellets were fixed in 10% buffered formalin overnight, embedded in paraffin, and sectioned (7 μm-thick). Differentiation into chondrocytes was assessed by evaluating the presence of glycosaminoglycans (GAG) after Alcian Blue staining (Pittenger, M. F. et al., 1999). Sections of mouse articular cartilage served as control.

Adipogenesis assay—Confluent MPCs were cultured for 10 days in DMEM low-glucose medium with 10% FBS, 1× GPS, and adipogenic supplements (5 μg/mL insulin, 1 μM dexamethasone, 0.5 mM isobutylmethylxanthine, 60 μM indomethacin). Differentiation into adipocytes was assessed by Oil Red O staining (Pittenger, M. F. et al., 1999).

Smooth muscle differentiation assay—cbEPCs were co-cultured with either bmMPCs or cbMPCs (1:1 EPCs to MPCs ratio) at a density of 2×10⁴ cell/cm² on FN-coated plates using EPC-medium. After 7 days, immunofluorescence was carried out using rabbit anti-human vWF (1:200; DakoCytomation, Carpinteria, Calif.), and mouse anti-human smooth muscle myosin heavy chain (1:100; Sigma-Aldrich) antibodies, followed by anti-rabbit TexasRed-conjugated, and anti-mouse FITC-conjugated secondary antibodies (1:200; Vector Laboratories). Vectashield with 4,6-diamidino-2-phenylindole (DAPI; Vector Laboratories) was used as mounting medium.

Indirect co-culture of cbEPCs and MPCs—cbEPCs were co-cultured with either bmMPCs or cbMPCs using a 0.4 μm Transwell-24 membrane culture system (Corning Incorporated Life Sciences, Acton, Mass.). cbEPCs were pre-cultured for 24 hours in the Transwell inserts at a density of 1×10⁴ cell/cm² after FN-coating using EPC-medium. Simultaneously, MPCs were pre-cultured separately onto FN-coated 24-wells at a density of 1×10⁴ cell/cm² using MPC-medium. After 24 hours, the top chambers where placed into the MPC wells and the resulting Transwell system cultured for 7 days using EPC-medium. Immunofluorescence was carried out using rabbit anti-human vWF (1:200; DakoCytomation), and mouse anti-human smooth muscle myosin heavy chain (1:100; Sigma-Aldrich) antibodies, followed by anti-rabbit TexasRed-conjugated, and anti-mouse FITC-conjugated secondary antibodies (1:200; Vector Laboratories). Vectashield with DAPI (Vector Laboratories) was used as mounting medium. SMCs served as control.

Measurement of VEGF in cell supematant—cbEPCs and MPCs were plated at a density of 2×10⁴ cell/cm² onto FN-coated 24-well plates using EPC- or MPC-medium respectively. After 24 hours, cells were washed and media replaced with DMEM containing 10% FBS, and 1× GPS and cell culture supernatant collected after 24 hours. Quantitative measurement of human VEGF in the cell culture supernatant was carried out using a Quantikine ELISA kit (R&D Systems, Minneapolis, Minn.). Values were normalized to total cell number determined at the time of supernatant collection.

Retroviral transduction of cbEPCs and bmMPCs—GFP-labeled cells were generated by retroviral infection with a pMX-GFP vector using a modified protocol from Kitamura et al., 1995 Proc Natl Acad Sci U S A 92, 9146-50. Briefly, retroviral supernatant from HEK 293T cells transfected with Fugene reagent and the vector was harvested, and both cbEPC and bmMPCs (1×10⁶ cells) were then incubated with 5 mL of virus stock for 6 hr in the presence of 8 μg/mL polybrene. GFP-expressing cells were sorted by FACS, expanded under routine conditions, and used for in vivo vasculogenic assays.

In vivo vasculogenesis assay—The formation of vascular networks in vivo was evaluated using a xenograft model as previously described above. Briefly, a total of 1.9×10⁶ cells was resuspended in 200 μl of ice-cold Phenol Red-free Matrigel™ (BD Bioscience, San Jose, Calif.), at ratios of 100:0, 80:20, 60:40, 40:60, 20:80 and 0:100 (EPCs:MPCs). The mixture was implanted on the back of a six-week-old male athymic nu/nu mouse (Charles River Laboratories, Boston, Mass.) by subcutaneous injection using a 25-gauge needle. Implants of Matrigel alone served as controls. One implant was injected per mouse. Each experimental condition was performed with 4 mice.

Histology and immunohistochemistry—Mice were euthanized at different time points and Matrigel implants were removed, fixed in 10% buffered formalin overnight, embedded in paraffin, and sectioned. Hematoxylin and eosin (H&E) stained 7 μm-thick sections were examined for the presence of lumenal structures containing red blood cells. For immunohistochemistry, 7-μm-thick sections were deparaffinized, and antigen retrieval was carried out by heating the sections in Tris-EDTA buffer (10 mM Tris-Base, 2 mM EDTA, 0.05% Tween-20, pH 9.0). The sections were blocked for 30 minutes in 5-10% blocking serum and incubated with primary antibodies for 1 hour at room temperature. The following primary antibodies were used: mouse anti-human CD31 (for human microvessel detection; 1:20; DakoCytomation, M0823 Clone JC70A; blocking with horse serum), goat anti-human CD31 (for CD31 and α-SMA co-staining; 1:20; Santa Cruz Biotechnology; blocking with rabbit serum), mouse anti-human α-SMA (1:750; Sigma-Aldrich; blocking with horse serum), rabbit anti-GFP antibody (1:4000; Abcam; blocking with goat serum), and mouse IgG (1:50; DakoCytomation; blocking with horse serum). Secondary antibody incubations were carried out for 1 hour at room temperature using either FITC- or TexasRed-conjugated specie-relevant antibodies (1:200; Vector Laboratories). For CD31 detection, biotinylated IgG/streptavidin-FITC conjugate (1:200; Vector Laboratories) incubations were carried out after primary antibodies. When double staining was performed, the sections were washed and blocked for 30 additional minutes in between the first secondary antibody and the second primary antibody. All the fluorescent-stained sections were counterstained with DAPI (Vector Laboratories). Projections of whole-mount GFP staining were performed on 100-μm-thick sections by confocal microscopy. Human infantile hemangioma, and mouse skin and lung served as control tissues. Additional staining was carried out with rabbit anti-human perilipin-A (1:750; Sigma-Aldrich, P1998) followed by FITC-conjugated rabbit antibody (1:200; Vector Laboratories). Additionally, horseradish peroxidase-conjugated mouse secondary antibody (1:200; Vector Laboratories) and 3,3′-diaminobenzidine (DAB) were used for detection of α-SMA, followed by hematoxilin counterstaining and Permount mounting.

Microvessel density analysis—Microvessels were quantified by evaluation of 10 randomly selected fields (0.1 mm each) of H&E stained sections taken from the middle part of the implants. Microvessels were identified as lumenal structures containing red blood cells and counted. Microvessels density was reported as the average number of red blood cell-filled microvessels from the fields analyzed and expressed as vessels/mm². Values reported for each experimental condition correspond to the average values ±S.D. obtained from at least four individual mice.

Luciferase assay—cbEPCs were infected with Lenti-pUb-fluc-GFP at a multiplicity of infection (MOI) of 10. The pUb-fluc-GFP was made based on the backbone of pHR-s1-c1a. The CMV promoter was replaced by the ubiquitin promoter, followed by a firefly luciferase/GFP fusion gene (Wu, J. C. et al., 2006, Proteomics 6, 6234-49). Lentivirus was prepared by transient transfection of 293T cells. Briefly, pUb-fluc-GFP was cotransfected into 293T cells with HIV-1 packaging vector and vesicular stomatitis virus G glycoprotein-pseudotyped envelop vector (pVSVG). Collected supernatant was filtered using a syringe filter (0.45 um) and concentrated by centrifuging at 5000 g for 2 hours. The virus was titrated on 293T cells. The infectivity was determined by GFP expression and luciferase/GFP-expressing cbEPCs were further sorted by fluorescence cytometry and used for in vivo vasculogenic assays. Luciferase/GFP-expressing cbEPCs were resuspended in 200 μl of Matrigel in the presence (40% cbEPC:60% bmMPCs) or absent of bmMPCs, at a total of 1.9×10⁶ cells. The mixture was implanted on the back of a six-week-old male nu/nu mouse by subcutaneous injection. One implant was injected per mouse. Each experimental condition was performed with 4 mice. At various intervals after implantation, the mice were imaged using an IVIS 200 Imaging System (Xenogen Corporation, Alameda, Calif.). Mice were anesthetized using an isofluorane chamber and were given the substrate, luciferin (2.5 mg/mL), by intraperitoneal injection according to their weights (typically 250 μl/30 gr). Bioluminescence was detected in implants 30-40 min after luciferin administration, and the collected data analyzed with Live Image 3.0 (Xenogen Corporation).

Cellularity of Matrigel implants—Mice were euthanized at different time points and Matrigel implants were removed, fixed in 10% buffered formalin overnight, embedded in paraffin, and sectioned. 7-μm-thick sections were deparaffinized and mounted with Vectashield with DAPI (Vector Laboratories). Cell nuclei were visualized using a fluorescent microscope, and counted in 4 randomly selected fields (0.29 mm2 each) of sections taken from the middle part of the implants. Cellularity was reported as the average number of nuclei from the fields analyzed and expressed as cells/mm2. Values reported for each experimental condition correspond to the average values ±S.D. obtained from at four individual mice.

Microscopy—Phase microscopy images were taken with a Nikon Eclipse TE300 inverted microscope (Nikon, Melville, N.Y.) using Spot Advance 3.5.9 software (Diagnostic Instruments, Sterling Heights, Mich.) and 10×/0.3 objective lens. All fluorescent images were taken with a Leica TCS SP2 Acousto-Optical Beam Splitter confocal system equipped with DMIRE2 inverted microscope (Diode 405 nm, Argon 488 nm, HeNe 594 nm; Leica Microsystems, Wetzlar, Germany) using either 20×/0.7 imm, 63×/1.4 oil, or 100×/1.4 oil objective lens. Non-fluorescent images were taken with a Axiophot II fluorescence microscope (Zeiss, Oberkochen, Germany) equipped with AxioCam MRc5 camera (Zeiss) using either 2.5×/0.075 or 40×/1.0 oil objective lens.

Statistical analysis—The data were expressed as means ±SD. Where appropriate, analysis of variance (ANOVA) followed by two-tailed Student's unpaired t-tests were performed. P value<0.05 was considered to indicate a statistically significant difference.

Results

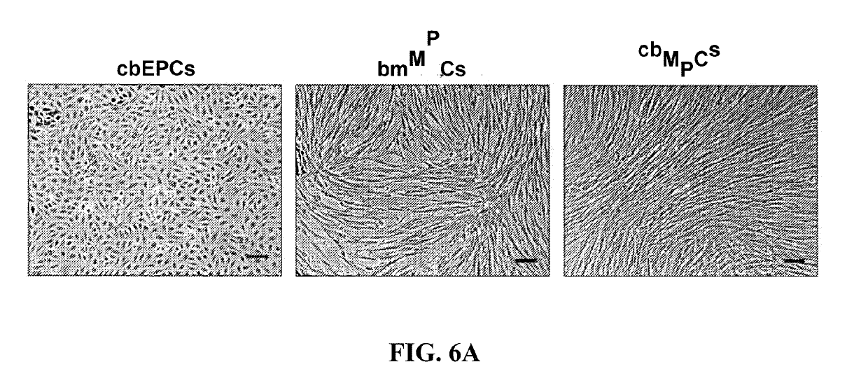

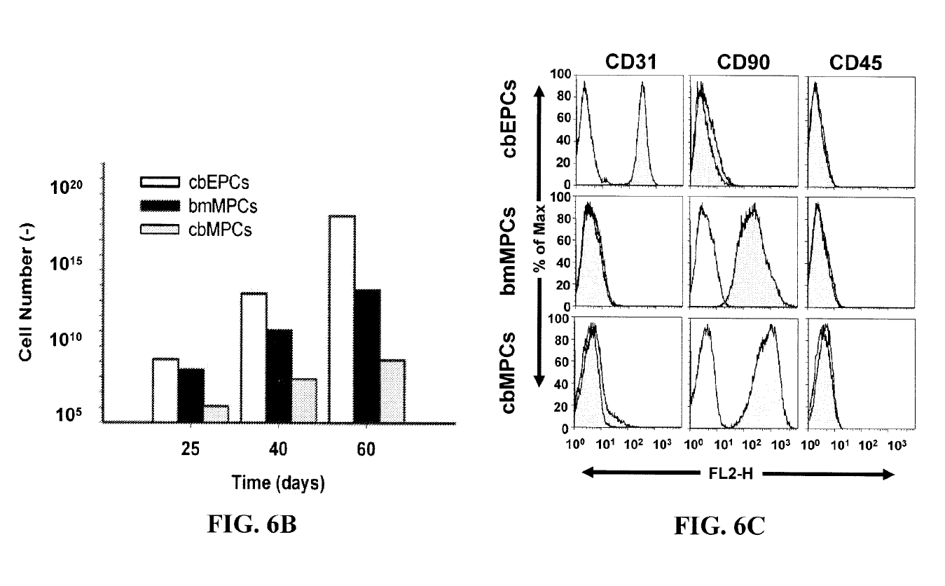

Isolation of endothelial and mesenchymal progenitor cells—Cord blood-derived EPCs (cbEPCs) (FIG. 6A) were isolated from the mononuclear cell (MNC) fraction of human umbilical cord blood samples and purified by CD31-selection as previously described supra. MPCs were isolated from the MNC fractions of both human bone marrow samples (bmMPCs) and human umbilical cord blood samples (cbMPCs). bmMPCs adhered rapidly to the culture plates and proliferated until confluent while cbMPCs emerged more slowly, forming mesenchymal-like colonies after one week. cbMPC colonies were selected with cloning rings and expanded. Both bmMPCs and cbMPCs (FIG. 6A) presented spindle morphology characteristic of mesenchymal cells in culture (Pittenger, M. F. et al., 1999).

cbEPCs and MPCs were grown in EPC-medium and MPC-medium respectively and their expansion potential estimated by the accumulative cell numbers obtained from 25 mL of either cord blood or bone marrow samples after 25, 40 and 60 days in culture (FIG. 6B). Remarkably, up to 10¹³ cbEPCs and 10¹¹ bmMPCs were obtained after only 40 days, which is consistent with previous data from example 1 and by Ingram, D. A., 2004. These values were further increased at 60 days, at which time 10¹⁸ cbEPCs and 10¹⁴ bmMPCs were estimated respectively. In the case of cbMPCs, a longer culture period was necessary to obtain a significant cell number (10⁹ cells). The reason for the apparent inferior potential of cbMPCs was likely due to the smaller number of MPCs in cord blood samples (typically 1-2 colonies every 25 mL; data not shown) as compared to bone marrow samples, where the majority of the adherent cells presented a distinctive mesenchymal morphology and contributed to the final bmMPC population.

The phenotypes of cbEPCs and MPCs were confirmed by three methods. Flow cytometry (FIG. 6C) showed that cbEPCs uniformly expressed the EC surface marker CD31, but not the mesenchymal and hematopoietic markers, as expected. Conversely, bmMPCs and cbMPCs showed uniform expression of the mesenchymal marker CD90 and were negative for CD31 and CD45. Western blot analyses confirmed the endothelial phenotype of cbEPCs (expression of CD31 and VE-cadherin) and the mesenchymal phenotype of bmMPCs and cbMPCs (expression of α-SMA and calponin) (data not shown). Indirect immunofluorescent staining indicate that bmMPCs and cbMPCs express mesenchymal markers α-SMA, calponin, and NG2 but not the EC markers CD31, VE-cadherin and vWF. Importantly, smooth muscle myosin heavy chain (smMHC), a specific marker of differentiated smooth muscle cells (Madsen, C. S. et al., 1998, Circ Res 82, 908-917; Miano, J. M., et. al., 1994, Circ Res 75, 803-812) was only found in mature SMCs but not in any of the MPCs.

The ability of MPCs to differentiate into different mesenchymal lineages was evaluated in vitro using well-established protocols (Pittenger, M. F. et al., 1999). Both bmMPCs and cbMPCs differentiated into osteocytes and chondrocytes as shown by the expression of alkaline phosphatase and GAG deposition in pellet cultures (data not shown). Adipogenesis was only evident with bmMPCs, but not with cbMPCs (data not shown). This loss of adipogenic potential of cbMPCs has been also reported for other mesenchymal cells in culture (Wall, M. E., et. al., 2007, Tissue Eng 13, 1291-8; Digirolamo, C. M. et al., 1999, Br J Haematol 107, 275-81) and was attributed to the more extensive expansion that these cells required due to their lower presence in cord blood samples.

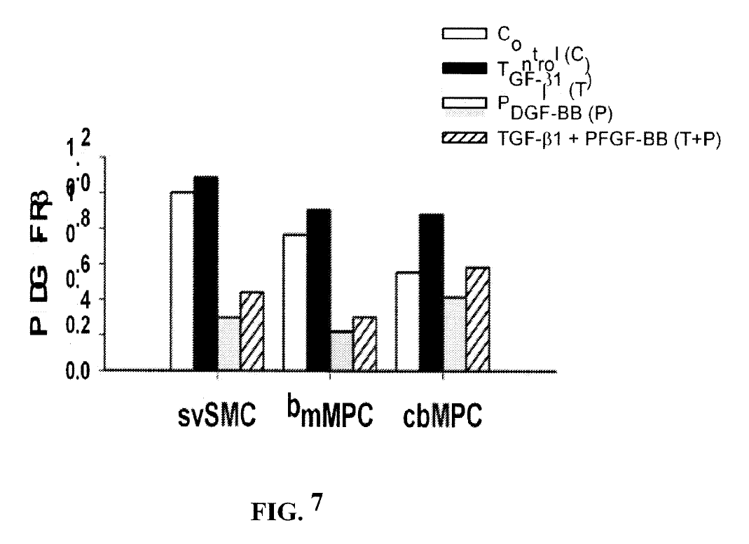

Since the MPCs were to be used as perivascular cells to engineer microvessel networks, the ability of MPCs to differentiate towards a smooth muscle phenotype was evaluated. As shown previously, both MPCs and mature SMCs shared a number of cellular markers including α-SMA, calponin, NG2, and PDGF-Rβ (FIG. 7). Although the definitive marker smMHC was absent in MPCs, both bmMPCs and cbMPCs were induced to express smMHC when directly co-cultured with cbEPCs (data not shown). Importantly, induction did not occur when MPCs were indirectly co-cultured with cbEPCs using a Transwell culture system, consistent with previous reports that showed direct contact between endothelial and mesenchymal cells is required for differentiation into SMCs (Antonelli-Orlidge, A., et. al., 1989, Proc Natl Acad Sci U S A 86, 4544-8).

In Vivo Formation of Human Vascular Networks

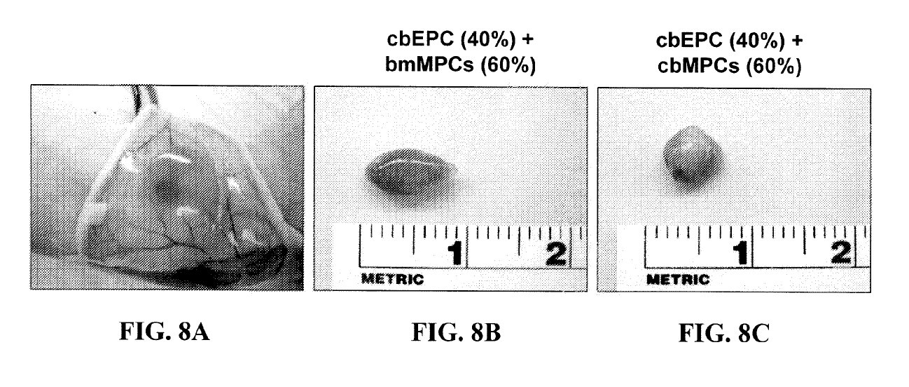

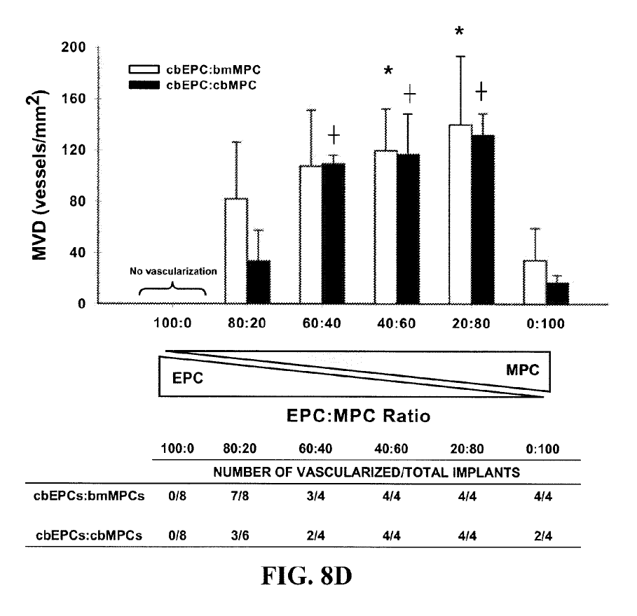

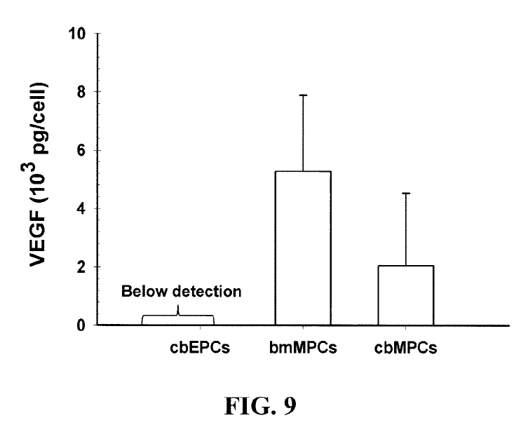

The vasculogenic capacity of blood-derived EPCs both in vitro and in vivo in example 1 and in Wu X., 2004. In these studies, the presence of vascular smooth muscle cells was crucial for formation of vascular networks. To answer the question of whether MPCs could act as alternative perivascular cells, different combinations of cbEPCs and MPCs (either bmMPCs or cbMPCs) were implanted into nude mice for one week (FIG. 8). A total of 1.9×10⁶ cells was resuspended in 200 μl of Matrigel, using ratios of 100:0, 80:20, 60:40, 40:60, 20:80 and 0:100 (% cbEPCs:% MPCs), and injected subcutaneously. After harvesting the Matrigel implants (FIG. 8A-C), H&E staining revealed numerous structures containing murine erythrocytes in implants containing both cbEPCs and MPCs (data not shown). The structures stained positive for human CD31 (data not shown), confirming the lumens were lined by the implanted cells. Implants of Matrigel alone were devoid of vessels indicating the Matrigel itself was not responsible for the presence of vascular structures. As shown in example 1, implants with cbEPCs alone failed to form microvessels after one week. Implants with only MPCs presented infiltration of murine blood capillaries, but no human microvessels (data not shown). The ability of MPCs to recruit murine vessels into Matrigel may be explained by the secretion of VEGF from MPCs but not cbEPCs (FIG. 9).

Quantification of microvessel density was performed by counting lumens with red blood cells (FIG. 8D) in implants with ratios of 100:0, 80:20, 60:40, 40:60, 20:80 and 0:100 (cbEPCs:MPCs; n≧4 each condition). The extent of the engineered vascular networks was highly influenced by the ratio of EPCs to MPCs (FIG. 8D). A progressive increase in MPCs resulted in increased microvessel density and higher frequency of vascularized implants (Table 1). When the ratio of EPC:MPC was 40:60, all implants were consistently vascularized at an average density of 119±33 vessels/mm² and 117±32 vessels/mm² with bmMPCs or cbMPCs respectively. These densities were significantly higher (P<0.05) than those observed with MPCs alone, reaffirming the necessity of the endothelial component for the formation of human vessels in the implants.

Assembly of Endothelial and Mesenchymal Progenitor Cells in the Vascular Bed.

In addition to the human CD31-positive lumenal structures, the engineered vessels were characterized by α-SMA staining of perivascular cells (data not shown). With either bmMPCs or cbMPCs, α-SMA-positive cells were detected both in the proximity and around the lumenal structures, indicating an ongoing process of perivascular cells recruitment for vessel maturation (Darland, D.C. & D'Amore, P. A., 1999, J Clin Invest 103, 157-158; Folkman, J. & D'Amore, P. A., 1996, Cell 87, 1153-1155; Jain, R. K., 2003, Nat Med 9, 685-693). In order to determine more precisely the contribution of each cell type, GFP-labeled cbEPCs were implanted with unlabeled MPCs. Anti-GFP staining clearly showed cbEPCs restricted to lumenal positions in the microvessel networks, while anti-α-SMA staining showed that the GFP-labeled vessels were covered by perivascular cells; this observation was valid with both sources of MPCs (data not shown). Projections of whole-mount staining showed that the GFP-expressing cells formed extensive networks throughout the implants (data not shown). Conversely, GFP-labeled bmMPCs were implanted with unlabeled cbEPCs to definitely identify input MPCs without relying on anti-α-SMA. Sections were stained with anti-GFP and anti-CD31 antibodies. In this experiment, GFP-expressing cells were detected as perivascular cells surrounding human CD31+lumens and as individual cells dispersed throughout the Matrigel implants (data not shown).

Durability of the Vascular Bed

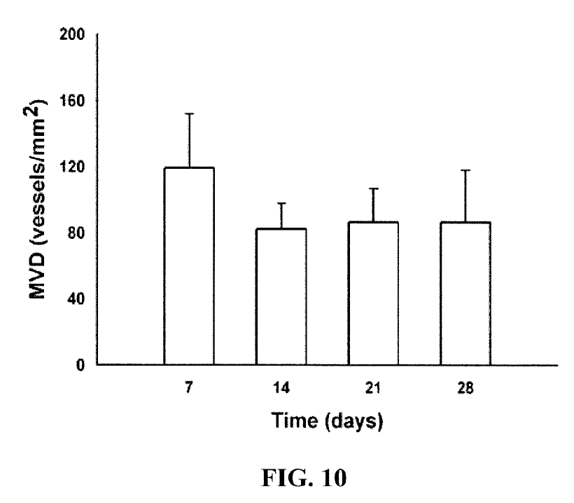

To test the durability of the engineered vascular beds in vivo, implants of cbEPCs-bmMPCs (40:60) were evaluated at 7, 14, 21 and 28 days after xenografting (FIG. 10). H&E staining revealed the presence of lumenal structures containing murine erythrocytes in all implants at each time point. Microvessel quantification revealed an initial reduction (statistically non-significant; P=0.105) in the number of patent blood vessels from 119±33 vessels/mm² at day 7 to 83±16 vessels/mm² at day 14. Microvessel densities remained stable thereafter (87±21 vessels/mm² and 87±32 vessels/mm² at days 21 and 28 respectively).

To further evaluate the durability of the engineered vascular bed in vivo, a luciferase-based imaging system was used to monitor perfusion of the Matrigel implants. cbEPCs were infected with lentivirus-associated vector encoding luciferase and implanted into immunodeficient mice in the presence or absence of bmMPCs. To assess perfusion, the mice were given the substrate, luciferin, by intraperitoneal injection at various time points after implantation. At 1 week, no bioluminescence was detected in implants with luciferase-expressing cbEPC alone, indicating that the substrate did not diffuse into the Matrigel. In contrast, a strong bioluminescent signal was detected in xenografts in which bmMPCs were co-implanted (data not shown). This result, coupled with parallel histological data, confirmed that the presence of MPCs was crucial to achieve rapid perfusion of the implants. Importantly, the luciferase-dependent signal was still detected 4 weeks after implantation, a further indication of the long-lasting nature of the engineered vessels.

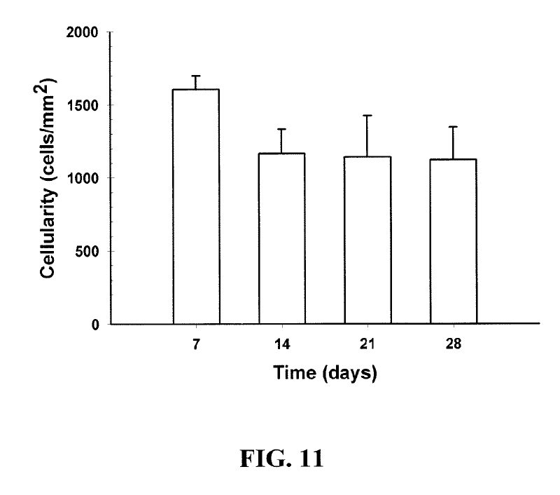

The cells within the Matrigel implants appeared to undergo a process of in vivo remodeling characterized by stabilization of total cellularity (FIG. 11) and redistribution of perivascular cells. α-SMA-expressing cells were initially detected (day 7) around the lumenal structures and throughout the Matrigel implants. However, over time the expression of α-SMA was progressively restricted to perivascular locations, as expected in normal stabilized vasculature (Jain, R. K., 2003). Finally, after 28 days in vivo, the presence of adipocytes was identified by staining with an anti-perilipin antibody (data not shown), indicating a process of integration between the implants and the surrounding mouse adipose tissue.

Vascular Network Formation Using Adult Progenitor Cells.

As with cbEPCs, it has been previously reported that adult peripheral blood-derived EPCs (abEPCs) are vasculogenic in vivo. However, when combined with mature SMCs at a ratio of 4:1 (EPC:SMC), implants using abEPCs required higher seeding densities in order to achieve similar microvessel densities to those obtained with cbEPCs as shown in example 1. This lower vasculogenic capacity of abEPCs has been reported recently by others (Au, P. et al., 2007, in press). The combination of adult bmMPCs and abEPCs at an optimized ratio (FIG. 8) would support the vasculogenic activity of abEPCs. Indeed, there are no previous reports on adult bmMPCs and abEPCs in the context of in vivo vasculogenesis. To evaluate this interaction, we isolated abEPCs as described in example 1, and confirmed their endothelial phenotype by inmunostaining of CD31, VE-cadherin and vWF (data not shown).

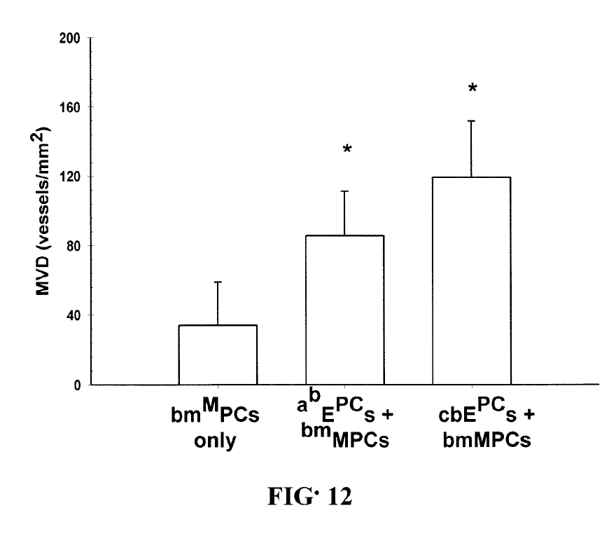

A total of 1.9×10⁶ cells (40% abEPCs and 60% bmMPCs) in Matrigel was implanted by subcutaneous injection into immunodeficient mice (FIG. 12). After harvesting the implants at 7 days, H&E staining consistently (n=4) showed an extensive presence of blood vessels containing murine erythrocytes. In addition, these lumenal structures stained positive for human CD31 (data not shown), confirming the lumens were formed by the implanted human abEPCs. Quantification of microvessel density (FIG. 12) revealed that the use of 40% abEPCs resulted in a statistically significant (P<0.05) increase in the number of blood vessels (86±26 vessels/mm²) as compared to implants with bmMPCs alone (34±25 vessels/mm²). Moreover, the difference between implants composed of abEPCs:bmMPCs and those of cbEPCs:bmMPCs (119±33 vessels/mm²) was not statistically significant (P=0.158), indicating that the presence of sufficient number of bmMPCs supported the vasculogenic properties of abEPCs to the same extent achieved with cbEPCs.

In conclusion, human postnatal EPCs and MPCs isolated from either blood or bone marrow have an inherent vasculogenic ability that can be exploited to create functional microvascular networks in vivo.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The references cited herein and throughout the specification are incorporated herein by reference.

1. A method of promoting neovascularization in a tissue in need thereof comprising contacting the tissue with a composition comprising an enriched population of isolated endothelial progenitor cells and an enriched population of isolated mesenchymal progenitor cells, wherein the endothelial progenitor cells and mesenchymal progenitor cells induce the formation of new blood vessels with functional connections to the host vasculature.

2. The method of claim 1, wherein the endothelial progenitor cells are derived from a source selected from a group consisting of bone marrow, cord blood, peripheral blood and blood vessel walls.

3. The method of claim 1, wherein the mesenchymal progenitor cells are derived from a source selected from a group consisting of amniotic fluid, bone marrow, cord blood, peripheral blood and adipose tissue.

4. The method of claims 2 or 3, wherein the progenitor cells are autologous to a recipient.

5. The method of claims 2 or 3, wherein the progenitor cells are HLA type matched to a recipient.

6. The method of claim 1, wherein the progenitor cells are both obtained from a sample of peripheral blood.

7. The method of claim 1, wherein the enriched populations of endothelial progenitor cells and mesenchymal progenitor cells are delivered simultaneously.

8. The method of claim 1, wherein the enriched populations of endothelial progenitor cells and mesenchymal progenitor cells are delivered sequentially.

9. The method of claim 1, wherein the tissue is a tissue engineered construct.

10. The method of claim 1, wherein the tissue is ischemic.

11. The method of claim 10, wherein the composition of progenitor cells is contacted by direct injection to the ischemic tissue or to healthy tissue adjacent to the ischemic tissue.

12. The method of claim 10, wherein the ischemic tissue is selected from a group consisting of the heart, skin, adipose tissue, muscle, brain, bone, liver, lungs, intestines, legs, limbs and kidneys.

13. The method of claim 1, wherein the enriched population of endothelial progenitor cells is at least 10% but not more than 90% of the composition.

14. The method of claim 1, wherein the enriched population of mesenchymal progenitor cells is at least 10% but not more than 90% of the composition.

15. The method of claims 13, wherein the endothelial progenitor cells is 40% of the composition.

16. A composition for promoting neovascularization comprising:
a. an enriched population of isolated endothelial progenitor cells;
b. an enriched population of isolated mesenchymal progenitor cells; and
c. a pharmaceutically acceptable carrier.

17. The composition of claim 16, wherein the composition is formulated for topical application.

18. The composition of claim 16, wherein the endothelial progenitor cells comprise at least 10% but not more than 90% of the total cells in the composition.

19. The composition of claim 16, wherein the mesenchymal progenitor cells comprise at least 10% but not more than 90% of the total cells in the composition.

20. The composition of claim 16, wherein the endothelial progenitor cells comprise about 40% and the mesenchymal progenitor cells comprise about 60% of the total cells of the composition.

21. The composition of claim 16, further comprising an extracellular matrix.

22. A kit comprising:
a. an enriched population of isolated endothelial progenitor cells; and
b. an enriched population of isolated mesenchymal progenitor cells.

23. The kit of claim 22, further comprising an extracellular matrix or a biocompatible scaffold.
